(19) United States
(12) Patent Application Publication
(10) Pub. No.: US 2023/0263927 A1
(43) Pub. Date: Aug. 24, 2023

(54) SYSTEM AND METHOD FOR DISINFECTING INDOOR ENVIRONMENTS

(71) Applicant: PURETEK GROUP INC, Boonton, NJ (US)

(72) Inventors: Michael Litvin, Boonton, NJ (US); Daniel Litvin, Boonton, NJ (US); Michael Tucker, Boonton, NJ (US); Tiago Zeitoune Geoffroy, Boonton, NJ (US)

(73) Assignee: PURETEK GROUP INC, Boonton, NJ (US)

(21) Appl. No.: 18/138,332

(22) Filed: Apr. 24, 2023

Related U.S. Application Data
(63) Continuation-in-part of application No. 17/494,126, filed on Oct. 5, 2021.
(60) Provisional application No. 63/087,379, filed on Oct. 5, 2020.

(51) Int. Cl. A61L 9/20

(52) U.S. Cl. CPC A61L 9/20; A61L 2209/15; A61L 2209/111; A61L 2209/12

(57) ABSTRACT

A system, method and apparatus for disinfecting an indoor environment. The system includes a user computer having a user app and a controller operably coupled with the user computer remotely through the user app. The user computer is capable of communicating with the controller through a network. The system further includes a fixture for housing the controller. Within the fixture are at least one UV-C device operably coupled with the controller, at least one motion sensor operably coupled with the controller, at least one fan operably coupled with the controller, and at least one air filter coupled with the at least one fan. The system further includes at least one air quality sensor. The at least one UV-C device and the at least one fan are operable in a plurality of modes controlled remotely by a user via the user app.

### CROSS-REFERENCE TO RELATED APPLICATION

This application is a continuation-in-part of U.S. application Ser. No. 17/494,126 titled “System and Method for Disinfecting Indoor Environments,” filed on Oct. 5, 2021, which claims priority to U.S. Provisional App. No. 63/087,379 titled “Disinfection System and Method,” filed on Oct. 5, 2020, which are incorporated by reference in their entireties.

### FIELD

The present invention relates to disinfection systems and methods, and in particular to a system and method for disinfecting indoor environments using ultraviolet radiation and simultaneously purifying air of indoor environments.

### BACKGROUND

Airborne infections spread when bacteria or viruses travel on dust particles or small respiratory droplets that become aerosolized when an infected person sneezes or coughs. Healthy people can inhale the infectious droplets, or the droplets can land on their eyes, nose and mouth.

One such airborne infection is COVID-19, which has caused many illnesses and deaths, and has affected daily activities of many. People are especially vigilant about spending time indoors because often times indoor air is not recycled with fresh outdoor air, and because many indoor environments have poor circulation. Thus, a need exists for disinfecting indoor environments to ensure that individuals are safe from contracting airborne infections.

One method of disinfecting indoor environments is by utilizing ultraviolet radiation (UVR), more specifically, UV-C, which is UVR with wavelengths between 100 and 280 nm. Currently, there are two distinct types concerning UV-C disinfection: Upper Room UV-C and Whole Room UV-C (UV-C Air and Surface). Such existing systems include, for example, Puro Lighting—Helo F1, Healthe Lighting—Cleanse Retrofit Troffer, Cooper Lighting Solutions—GSL Germicidal UV Striplight and American Ultraviolet—TB, RAM Series.

Upper Room UV-C systems treat the room during periods of occupancy but are limited to a low threshold limit value. As such, even though Upper Room UV-C systems can function in occupied spaces, their efficacy in deactivating pathogens is limited by an output threshold limit value not to exceed 6.0 mJ/cm2 at 254 nm by the American Conference of Governmental Industrial Hygienists (ACGIH) Committee on Physical Agents. This threshold limits the efficacy of Upper Room UV-C systems with research showing effectiveness not exceeding 80%, as shown at https://stacks.cdc.gov/view/cdc/11285.

Whole-Room UV-C systems operate at much higher output. Whole Room UV-C disinfection systems have shown 99.9% or higher inactivation rates of pathogens, as shown at https://www.ncbi.nlm.nih.gov/pmc/articles/PMC5369231/. However, Whole Room UV-C disinfection systems should only be activated during periods of non-occupancy as excessive exposure can lead to corneal irritation and skin reddening and irritation (photodermatitis).

The dose delivered by a particular product is based on the UV-C irradiance and the duration of exposure. As a result, 4-log reductions of aerosolized viruses, bacteria, and fungi were achieved at dosages of 25 mJ/cm2 or less in a Whole Room UV-C treatment test, as shown at https://aem.asm.org/content/84/17/e00944-18.

Therefore, a need exists for a hybrid Upper Room and Whole Room UV-C system and method that maximizes the air and surface disinfection benefits of both existing systems for operation during occupancy and non-occupancy.

While UV-C lighting fixtures exist, they are designed as a one-size-fits-all and are configured to be mounted on a ceiling without regard for the height of the ceiling. As a result, many UV-C lighting fixtures in the prior art do not accommodate for lower ceilings and smaller spaces. Therefore, a need exists for UV-C lighting fixtures adaptable for lower ceilings and smaller spaces.

UV-C lighting fixtures in the prior art are also specifically geared toward disinfecting air and surfaces but do not provide air purification. As a result, a user is required to have a fixture for disinfecting air and surfaces via UV-C, and a separate fixture or device for air purification. Therefore, a need exists for a single fixture or device capable of both UV-C disinfection and air purification.

Control systems relating to air disinfection and purification are available in the market. Examples of such are Atmos Air, Aura Air, rZero, Acuity nLight, airThinx, Awair and Magectech. However, those systems have numerous shortcomings. First, some of the existing systems solely monitor air without providing any means of treatment, thus requiring the user to install and integrate separate treatment systems. Second, some of the existing systems provide treatment without any feedback to efficacy or real-time air quality. As a result, the systems are inefficient, unscientific, and, therefore, potentially inefficacious. Third, unlike UVC disinfection systems which have been tested, refined and proven for over 100 years, the systems of the prior art are focused on treatment with unproven technology. Often times prior art systems use unproven and dubious technologies with improper placement of sensors to manipulate data readings. Fourth, existing control systems lack user engagement and make data exclusive to a few authorized system users. Also, there are no visible Indoor Air Quality (IAQ) monitors or guest access, which limits system efficacy, reduces opportunities for building administration to promote the system, cuts down on wellbeing improvements to occupants, and eliminates transparency. Fifth, prior art systems are not designed specifically for air quality improvements. In those systems, control protocols are designed for other non-essential functions resulting in a convoluted and difficult-to-use system. Without being inherently designed for the purpose of monitoring and performing disinfection and purification, those systems are unreliable and expansion is not guaranteed. Therefore, a need exists for a system for monitoring and treating air quality via live feedback loop for IAQ and UVC technologies by incorporating multiple means of system engagement, i.e., local IAQ monitor, web application and smart phone application, for varying users including building occupants.

### SUMMARY

The following presents a simplified summary of some embodiments of the invention in order to provide a basic understanding of the invention. This summary is not an extensive overview of the invention. It is not intended to identify key/critical elements of the invention or to delineate the scope of the invention. Its sole purpose is to present some embodiments of the invention in a simplified form as a prelude to the more detailed description that is presented later.

The purpose of the present invention is to provide an automated and comprehensive disinfection of pathogens in facilities during periods of occupancy and non-occupancy. The advantage of a hybrid UV-C system and method of the present invention that emits short-term highly effective treatments via Whole-Room operation and long-term treatments during periods of occupancy via Upper Room operation is very clear. The system and method of the present invention will not only provide high effective UV-C dosage treatments during periods of occupancy and vacancy, but of even more importance, the hybrid UV-C system and method of the present invention, on account of combining continual long-term and short-term treatments, maintain dosage values that come as close to 4-log or greater reductions that is currently available on the market today.

The hybrid Upper Room and Whole Room UV-C disinfection system and method of the present invention functions in two separate Upper Room and Whole Room treatment modes. Upper Room treatment occurs during periods of occupancy and Whole Room treatment occurs during periods of non-occupancy. In one embodiment, the Upper Room and Whole Room operation is controlled via an app-based (iOS and Android) Bluetooth mesh control system that is integrated into the overall system of the present invention. In another embodiment, the operation is be controlled via manual wall switch for Upper Room and an app-based (iOS and Android) Bluetooth mesh control system that is integrated into the overall system of the present invention for Whole Room.

To achieve the above-mentioned purpose, the present invention provides a UV-C lighting fixture equipped with various components including a controller that is operably coupled with a user app accessible by a user through a user device. The fixture is programmed by the user to operate the fixture during occupancy and/or vacancy to maximize disinfection of interior environments in a safe manner.

The present invention also provides a system for monitoring and treating air quality via live feedback loop for IAQ and UVC technologies by incorporating multiple means of system engagement, i.e., local IAQ monitor, web application and smart phone application, for varying users including building occupants. Unlike other systems in the prior art that provide partial solutions, the system of the present invention uniquely provides a holistic approach by combining various features in one system. Live air quality monitoring provides scientific identification of dangerous gases, particulates, and viral index in real time. Healthy air score provides users quick confirmation of level of indoor air quality. Sensor arrays are placed as per ASHRAE recommendations to provide validated and non-biased data. Automated responsive treatment via live feedback loop ensures high level air quality while limiting energy usage, expanding device lifespan, and making a truly human-centric solution. UVC-based technologies of the system of the present invention provide over 100 years of tested and validated disinfection of dangerous pathogens. In addition, the control system flexibly incorporates additional research-proven technologies including HEPA, active carbon filtration, air circulation, and far UVC. Also, the present invention provides direct system engagement opportunities for all occupants of a facility including guests ensure validation, education and confidence in the air that all a facility's occupants breathe. Digital IAQ monitors and applications launched via scannable QR codes provide engaging and interactive data. Approved users have access to a wide range of tools to maximize system usage including printable/sharable reports, historic in-depth data, component replacement notifications and more. Software upgrades and IoT-ready components ensure a flexible and future-ready solution that can address tomorrow's indoor air quality needs as well as new advancements in building technologies. Lastly, live monitoring of component health via power reading, air flow rate, etc. with an automatic option to source replacement components is provided by the present invention.

In one aspect, the present invention provides a system for disinfecting an indoor environment, the system comprising: a user computer having a user app; a controller operably coupled with the user computer remotely through the user app, the user computer capable of communicating with the controller through a network; a fixture for housing the controller, the fixture further comprising: at least one UV-C device operably coupled with the controller, at least one motion sensor operably coupled with the controller, at least one fan operably coupled with the controller, and at least one air filter coupled with the at least one fan; and at least one air quality sensor; wherein the at least one UV-C device and the at least one fan are operable in a plurality of modes controlled remotely by a user via the user app.

In another aspect, the present invention provides a method for disinfecting an indoor environment, the method comprising the steps of: programming, by a user computer through a user app located remote from the user computer, parameters for controlling at least one UV-C device and at least one fan having an air filter; communicating the parameters to a controller, through a network, the controller operably coupled with the at least one UV-C device and a motion sensor for detecting occupancy within the indoor environment, and the at least one fan and an air quality sensor for measuring air quality within the indoor environment; storing, in a memory, the parameters on the controller; and controlling, by a processor, the at least one UV-C device and the at least one fan for operation in a first mode when the indoor environment is occupied, a second mode when the indoor environment is unoccupied, and a third mode when air quality parameters are not met; wherein in the first mode, the at least one UV-C device is activated by the controller to emit ultraviolet radiation to a first section of the indoor environment at a first level, in the second mode the at least one UV-C device is activated by the controller to emit ultraviolet radiation to sections of the indoor environment beyond the first section of the indoor environment at a second level, and in the third mode the at least one fan is activated.

In yet another aspect, the present invention provides an apparatus for disinfecting an indoor environment, the apparatus comprising: a controller operably coupled with a remote user computer having a user app, the user computer capable of communicating with the controller via a network; at least one UV-C device operably coupled with the controller; at least one motion sensor operably coupled with the controller; and at least one fan operably coupled with the controller; wherein the at least one UV-C device and the at least one fan are operable in a plurality of modes controlled remotely by a user via the user app.

### BRIEF DESCRIPTION OF DRAWINGS

The foregoing summary, as well as the following detailed description of presently preferred embodiments of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIG. 1 is a flow chart illustrating the two modes of operation of an embodiment of a system and method of the present invention;

FIG. 2 is a front perspective view of an embodiment of a fixture of the present invention with a front-facing grill of the fixture removed;

FIG. 3 is a front perspective view of the fixture of FIG. 1 in an open state;

FIG. 4 is a front perspective view of the fixture of FIG. 1 in a partially open state;

FIG. 5 is a front perspective view of the fixture of FIG. 1 in a closed state;

FIG. 6 is a front perspective view of another embodiment of a fixture of the present invention;

FIG. 7 is a bottom perspective view of the fixture of FIG. 1 in a closed state;

FIG. 8 is a wiring diagram of an embodiment of a controller of the present invention;

FIG. 9 is a flow chart illustrating the two modes of operation of another embodiment of a system and method of the present invention;

FIG. 10 is a bottom side perspective view of another embodiment of a fixture of the present invention;

FIG. 11 is a bottom perspective view of the fixture of FIG. 10 with an outer casing superimposed on the fixture;

FIG. 12 is another bottom side perspective view of the fixture of FIG. 10 with an upper lamp illuminated;

FIG. 13 is a bottom view of the fixture of FIG. 10 with the upper lamp illuminated;

FIG. 14 is a bottom view of the fixture of FIG. 10 with a lower lamp illuminated;

FIG. 15 shows mounting components for the fixture of FIG. 10;

FIG. 16 is a wiring diagram of the fixture of FIG. 10;

FIG. 17 is a top side perspective view of another embodiment of a controller of the present invention;

FIG. 18 is a wiring diagram of the controller of FIG. 17;

FIG. 19 shows an embodiment of a sensor of the present invention;

FIG. 20 shows another embodiment of a sensor of the present invention;

FIG. 21 is an illustration of a motion detection range for the sensor of FIG. 19;

FIG. 22 is illustration of a motion detection range for the sensor of FIG. 20;

FIG. 23 is an overall schematic of the system of the present invention;

FIG. 24 is a schematic of the user app of the present invention;

FIG. 25 is a perspective view of another embodiment of a portable fixture of the present invention;

FIG. 26 is an exploded view of an alternative embodiment of a portable fixture of the present invention;

FIG. 27 is a bottom perspective view of another embodiment of a fixture of the present invention;

FIG. 27A is a perspective view of internal components of the fixture of FIG. 27;

FIG. 28 is a bottom view of another embodiment of a fixture of the present invention;

FIG. 29 is an illustration of internal components of the fixture of FIG. 28;

FIG. 30 is a flow chart illustrating an embodiment of the system of the present invention;

FIG. 31 is an illustration of a display of the software of the present invention in use;

FIG. 32 is an illustration of an IAQ monitor display of the present invention;

FIGS. 33 and 34 are an illustrations of user screens of the software of the present invention.

To facilitate an understanding of the invention, identical reference numerals have been used, when appropriate, to designate the same or similar elements that are common to the figures. Further, unless stated otherwise, the features shown in the figures are not drawn to scale and are shown for illustrative purposes only.

### DETAILED DESCRIPTION

Certain terminology is used in the following description for convenience only and is not limiting. The article “a” is intended to include one or more items, and where only one item is intended the term “one” or similar language is used. Additionally, to assist in the description of the present invention, words such as top, bottom, side, upper, lower, front, rear, inner, outer, right and left are used to describe the accompanying figures. The terminology includes the words above specifically mentioned, derivatives thereof, and words of similar import.

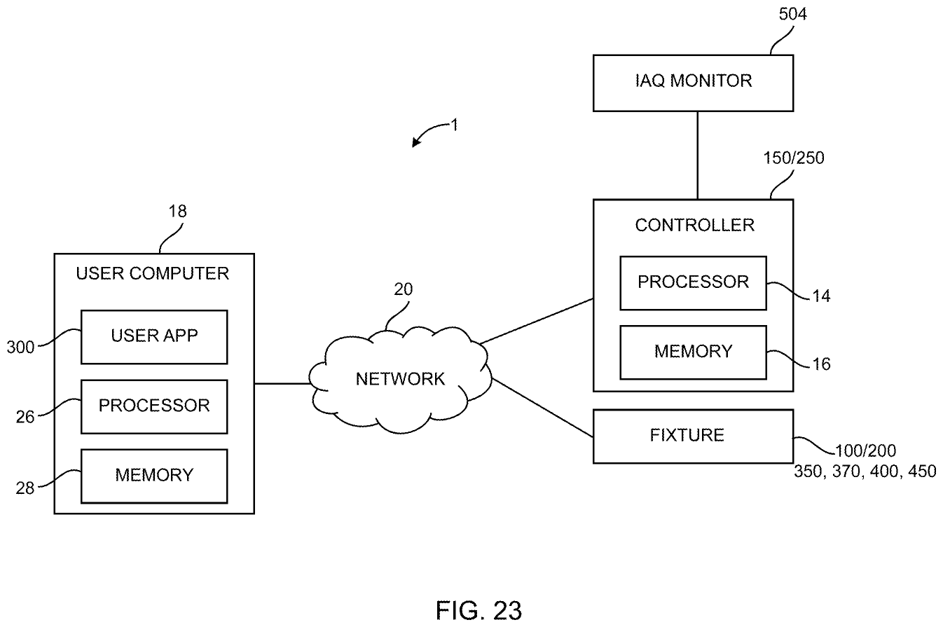

In general, as shown in FIG. 23, the system 1 of the present invention includes a fixture, e.g., 100, 200, 350, 370, 400, 450 having a controller, e.g., 150, 250, that communicates with a user device or computer 18 via a user app 300. The user app 300 is downloaded on a user computer 18 having a processor 26 and memory 28, with the user computer 18 connected to a network 20. The controller, e.g., 150, 250, which controls various functions of the fixture 100, 200, 350, 370, 400, 450 also includes a processor 14 and memory 16. The controller, e.g., 150, 250, is programmed through the user app 300 for various parameters. The network 20 could be any one of or a combination of a wireless network such as 4G, 5G, Wi-Fi or Bluetooth, or hard wired. Even though FIG. 23 is shown with only one controller 150, 250 coupled to the user app 300 via the network 20, multiple controllers could be coupled thereto such that a user is able to control multiple controllers within the same network.

### First Fixture Embodiment

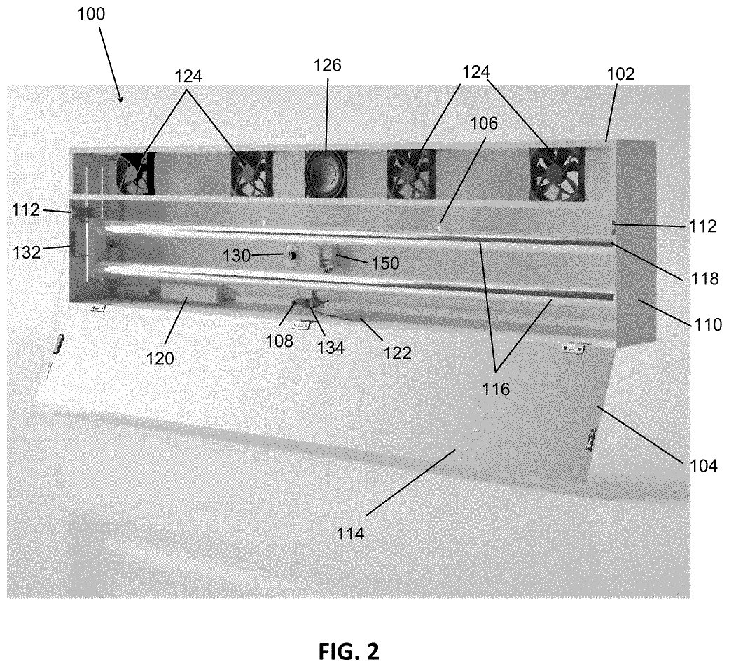

Referring to FIGS. 2-7, in one embodiment, the fixture 100 includes a housing 102 and an automated adjustable shield 104 that is pivotably coupled to the housing 102. In this embodiment, the housing 102 is constructed of 22-gauge white matte steel with a high reflectance polyester powder coat finish. Referring to FIG. 2, two pre-drilled openings 106 are provided on the housing 102 for securing the fixture 100 to a wall or ceiling. In this embodiment, each opening 106 is ¼ inch and the pair openings 106 are 16 inches apart to accommodate standard stud spacing. One of ordinary skill in the art would recognize that the openings 106 could be spaced apart at other lengths, for example, 24 inches, or further openings could be provided to accommodate for alternative mounting. For example, the housing 102 could be provided with threaded openings on a top portion for optional trunnion ceiling mount.

As well, as shown in FIG. 2, two % inch knockouts 108 are provided at or about the center rear of the housing 102 for power entry. The fixture 100 and its components are powered by electricity provided by a local utility, for example, 110V-277V/60 Hz AC in the U.S. However, the fixture 100 and its components could be configured to operate under other voltages and frequencies. Optionally, 120V and 277V cord and plug accessories could be provided for users who prefer not to hard-wire the fixture 100. A safety interlock switch 132 is provided to isolate power when the fixture 100 is manually opened. A power distribution block 134 is also provided for power and control wiring connections.

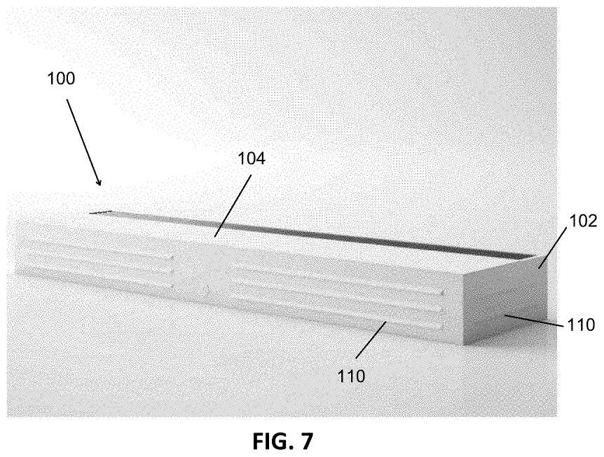

As shown in FIG. 2, two aluminum air intake grills 110 are provided on each side of the housing 102. The intake grills 110 allow for air to enter into the fixture 100. Additional intake grills 110 are provided on a bottom portion of the housing 102, as shown in FIG. 7, to further promote air flow.

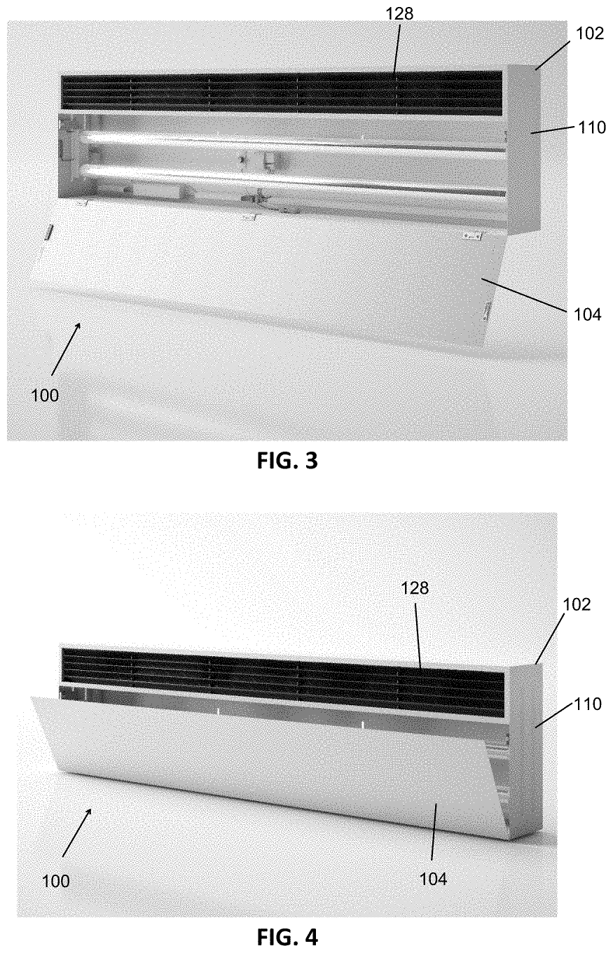

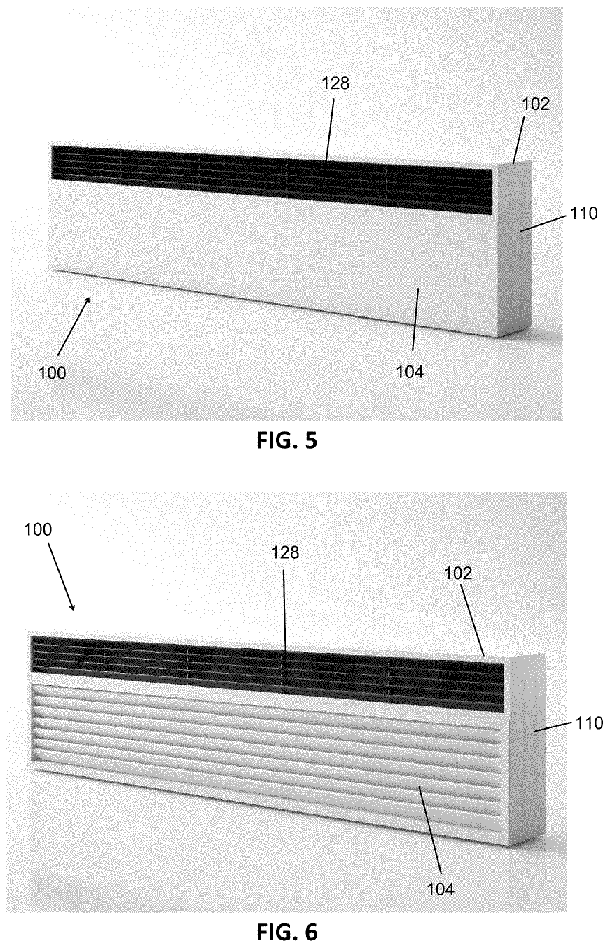

Still referring to FIG. 2, the shield 104 is operably coupled to and driven by a shield motor mechanism 112 on each side of the shield 104 such that the shield 104 is pivotable between a closed state as shown in FIGS. 5-7 when in Upper Room disinfection mode, a partially closed state as shown in FIG. 4, and an open state as shown in FIGS. 2 and 3 when in Whole Room disinfection mode. The shield 104 includes a polished specular aluminum reflector 114 on an inner surface to reflect UV-C light when in operation. The shield 104 prevents eye exposure to UV-C irradiation during the Upper Room treatment mode when the fixture 100 is in a closed state. Optionally, the shield 104 could be grated as shown in FIG. 6 to provide additional air circulation.

Still referring to FIG. 2, various components of the fixture 100 are secured to the interior of the housing 102. Two 46″ UV-C lamps 116 are mounted to UV-C resistant ceramic bi-pin lamp holders 118. In this embodiment, the UV-C lamps 116 are low pressure mercury UV-C lamps with specifications as follows—(i) lamp wattage: 37.9 W; (ii) lamp current: 0.420±0.040 A; (iii) lamp voltage: 106V; (iv) tube length (L): 1,152.0±1.3 mm; (v) tube length (P-P): 1168.0 (max.); (vi) tube diameter (D): 32.5±1.55 mm; (vii) base: G13; (viii) spectral peak: 253.7 nm; (ix) UV output: 18.0 W; (x) average life: 8,000 hrs; (xi) ballast: F40T10 or G40T10; and (xii) glow starter: FG4P (JIS). The UV-C lamps 116 are operably coupled to an electronic ballast 120 by wire. In this embodiment, the ballast 120 is suitable for 80 W 2-lamp load, operational for 100-277 VAC and rated for duty cycling. As well, the electronic ballast 120 is coupled by wire to a UL-listed luminaire disconnect plug 122 to connect to input power wiring for maintenance, in compliance with the National Electrical Code (NEC). Alternatively, custom sized amalgam UV-C lamps, described below, could be used to provide longer lifespans as a result of operating at a more pressurized state.

Referring again to FIG. 2, four frictionless fans 124 and an audible alarm speaker 126 are operably mounted to an upper section of the housing 102 and covered by a front-facing grill 128, as shown in FIGS. 3-6. In this embodiment, the front-facing grill 128 is constructed of rigid plastic and painted black.

In this embodiment, the fans 124 are mounted on a narrow platform that extends horizontally from opposite sides of the fixture 100. With this configuration, a space is formed between the back portions of the fans 124 and a rear wall of the fixture 100. As a result, an unimpeded an upward air stream is formed within the fixture 100 is formed when the fans 124 are activated. Each fan 124 pulls outside air through the air intake grills 110 and pushes air out through the front-facing grill 128. The purpose of the fans 124 is twofold. First, the fans 124 pull room air in through the side vents 110 in order to clean the air and expel it through the front grill 128. Second, the fans 124 improve air mixing within the room which will improve UV-C treatment efficacy. Fan speed can be adjusted via the user app 300, which applicant provides under the trademark Intelli-Safe, which communicates with a controller 150, as will be described in more detail below.

The speaker 126 plays back pre-recorded messages during activation and operation of the fixture 100. For example, in the Whole Room treatment mode, as an added layer of protection, a non-invasive yet clearly audible message is emitted at 65 db to notify occupants to evacuate the treatment area in order to avoid being exposed to Whole Room UV-C irradiation. As well, an audible alert signals transitioning from Upper Room to Whole Room treatment modes and warns occupants to evacuate when activating Whole Room treatment.

One of ordinary skill in the art would recognize that the number and size of the fans 124 and the speaker 126 could be modified without departing from the spirit and scope of the invention.

As shown in FIG. 2, an RF motion sensor 130 is also mounted to the housing 102 and operably coupled with the controller 150. The integrated RF motion sensor 130 can detect occupants within the treatment area and maintain Upper Room treatment and/or disengage operation as per programming. In this embodiment, Whole Room treatments will only begin when the RF sensor 130 does not detect any motion for a pre-programmed time i.e. 5 minutes, but other time limits could be set as desired.

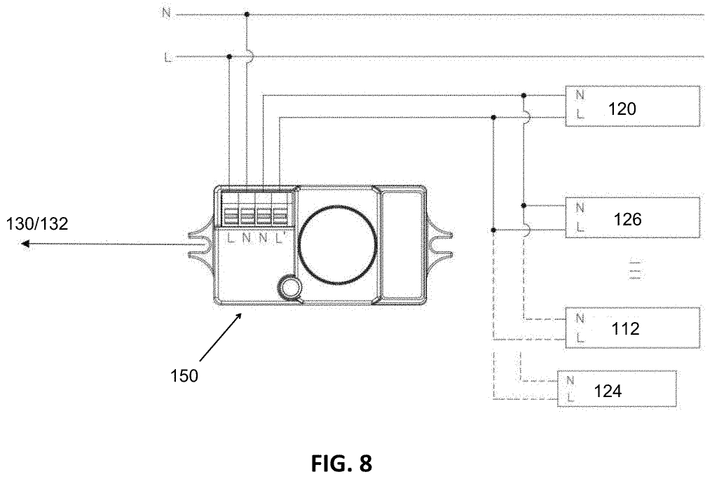

Referring to FIG. 8, the controller 150 is operably coupled to the other components of the fixture 100 by wiring with the electronic ballast 120, alarm speaker 126, shield motors 112 and fans 124 on one end, and operably coupled by wire to low voltage connections, i.e., the RF motor sensor 130 and safety interlock switch 132, on another end. The controller 150 is programmed and controlled by the user via the user app 300. In this embodiment, the user app 300 operates on iOS and Android on a mobile device. However, the user app 300 could also be used on desktop and laptop computers.

The controller 150 and the user app 300, collectively, is a Bluetooth mesh control system that controls the fixture 100 by executing pre-programmed and on-demand automated disinfection in order to ensure that Upper Room and Whole Room treatment modes are in accordance with the various features, including the exemplary features outlined above and explained in more detail below. The control system, i.e., the controller 150 and user app 300, which applicant markets under the trademark Intelli-Safe, includes many features that aid in safely disinfecting an interior environment. A remote monitoring wireless hub may be provided with the system 1 of the present invention so that the control system is connected to the user's computer network 20. As such, the user has the ability to remotely monitor status and operation of the hybrid fixtures 100.

### Operation of System with First Fixture Embodiment

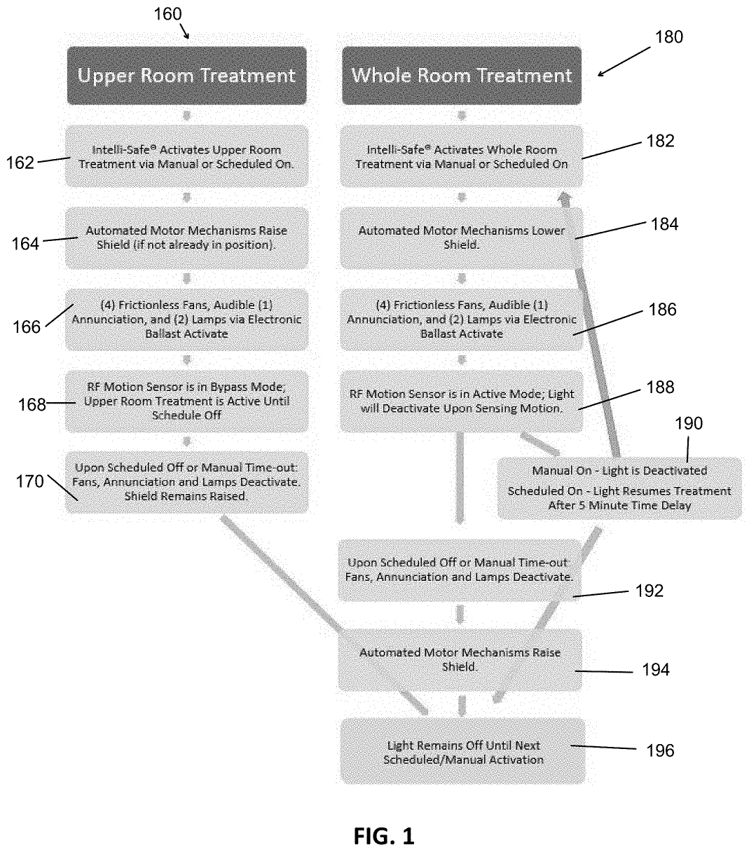

Referring to FIG. 1, in operation, once the Upper Room treatment mode 160 has been initiated via the Intelli-Safe controls (Step 162), the automated shield 104 will rise (Step 164) when switching from Whole Room mode or remain in the closed position, as shown in FIG. 5, when activating from a deactivated state. The two UV-C lamps 116 will be on along with the fans 124 (Step 166). Preferably, the fixture 100 operates at a wavelength of 254 nm but the wavelength could be varied based on utilized UV lamp technology. In addition, the speaker 126 will announce any messages that are programmed. In order to prevent any UV-C irradiation from direct eye visibility, fixtures 100 are mounted at no less than 7 ft heights from the floor in order to ensure that output threshold limits as a result of direct eye exposure are not exceeded. During Upper Room treatment, the motion sensor 130 is in bypass mode and the Upper Room treatment is active until the scheduled time to turn off (Step 168). Upon the termination of the Upper Room treatment, the lamps 116, fans 124 and speaker 126 deactivate while the shield 104 remains raised such that the fixture 100 is in a closed state (Step 170). Each of these components remain off until the next scheduled activation or manual activation (Step 196).

Still referring to FIG. 1, when Whole Room treatment 180 is initiated via the Intelli-Safe controls (Step 182) the automated shield 104 will lower (Step 184), as shown in FIG. 3, and a timed treatment will occur in accordance with the pre-programmed settings. The two UV-C lamps 116 will be on along with the fans 124 (Step 186). In addition, the speaker 126 will announce any messages that are programmed and the motion sensor 130 will be in active mode. The Hybrid UV-C fixture 100 will turn off once the Whole Room timed treatment ends in accordance with the Intelli-Safe programming. As well, if any motion is detected by the motion sensor 130 during the Whole Room treatment, then the fixture 100 will immediately deactivate for a preset period of time during which no motion is sensed (Step 188). During this situation, once the motion time-out period expires (Step 190), the fixture 100 will resume Whole Room mode (Step 182) to finish the current treatment or deactivate in accordance with the controller 150 programming. Upon the termination of the Upper Room treatment, the lamps 116, fans 124 and speaker 126 deactivate (Step 192) while the shield 104 is raised (Step 194) such that the fixture 100 is in a closed state. Each of these components remain off until the next scheduled activation or manual activation (Step 196).

### Second Fixture Embodiment

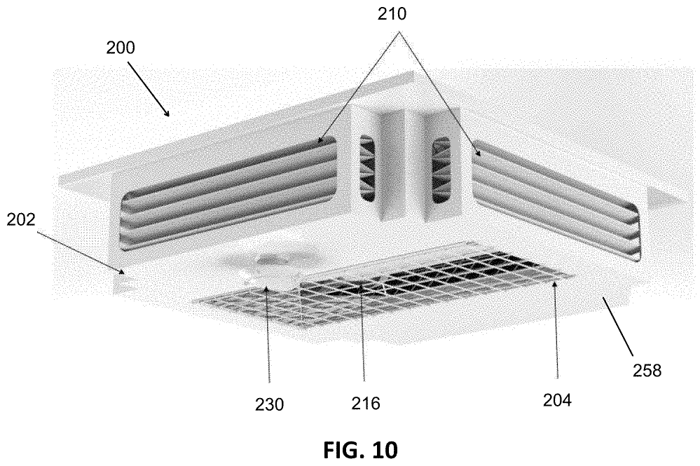

Referring to FIGS. 10-20, in another embodiment, the fixture 200 includes a housing 202 enclosed with an outer casing 258. In this embodiment, the housing 202 is constructed of 304/316 stainless steel with a polyester powder coat finish. The exterior finish is shown as white but can also be a black finish or customized color. In this embodiment, the overall dimensions of the fixture 200 are 603 mm (L)×603 mm (W)×255 mm (H). The fixture 200 and its components are powered by electricity provided by a local utility. The fixture 200 includes two (2) 300 W rated electronic ballasts 220, which are rated for 110V-277V/60 Hz AC to power corresponding 300 W UV-C lamps 216a, 216b, which are stacked against each other vertically. However, the fixture 200 and its components could be configured to operate under other voltages and frequencies. Optionally, 120V and 277V cord and plug accessories could be provided for users who prefer not to hard-wire the fixture 100.

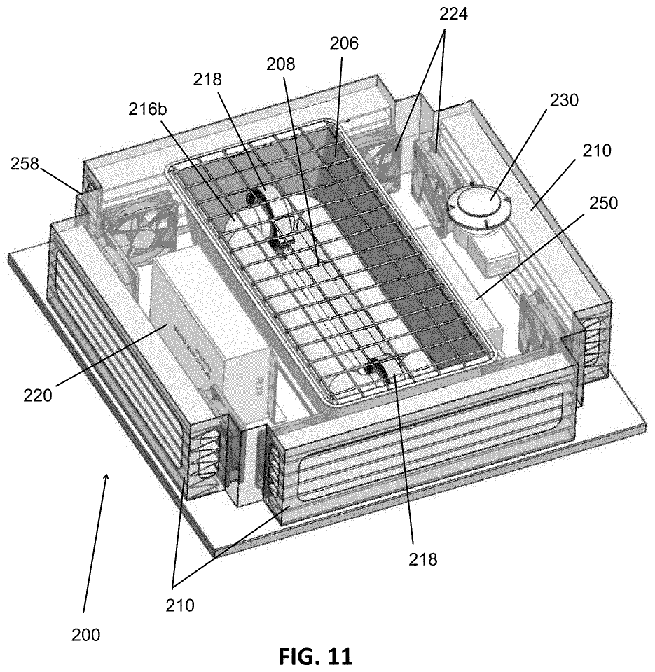

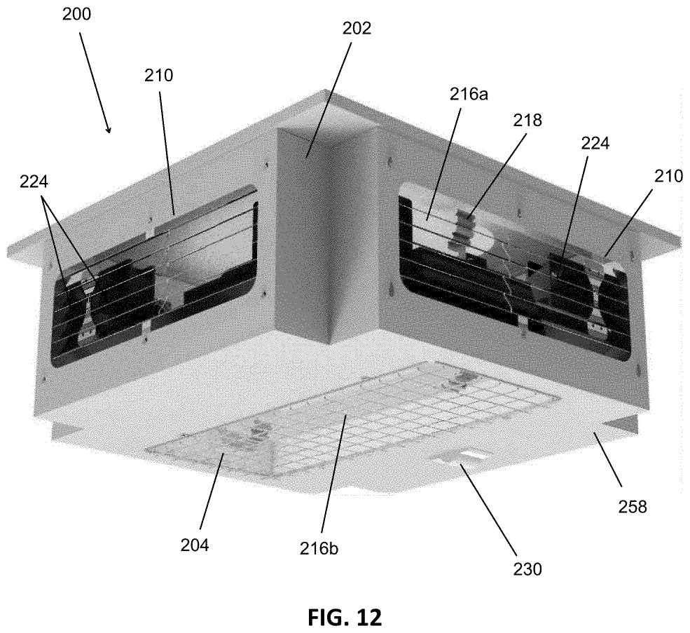

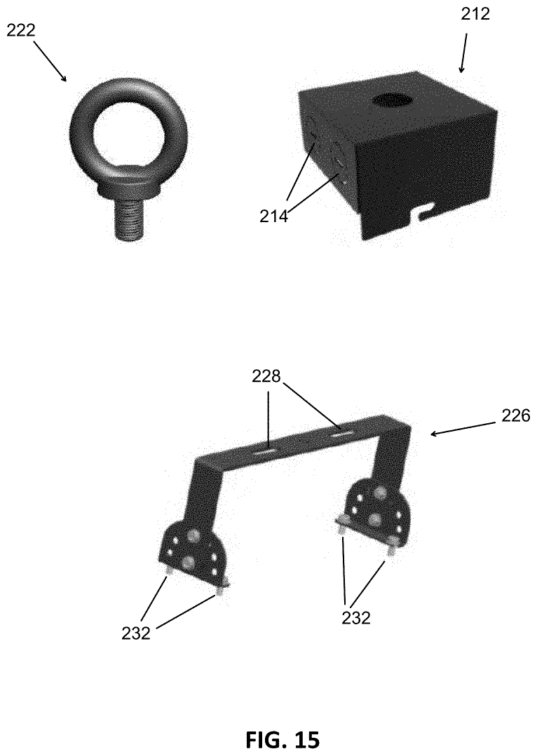

As shown in FIGS. 10-14, the fixture 200 includes a stainless-steel wire guard 204 to protect the lower UV-C lamp 216b from physical damage. A disinfection chamber 206 is provided to shield any UVC light from Upper Room operation, described in more detail below, from emitting downwards to avoid exposure. That is, a shown in FIGS. 11 and 12, the three-sided disinfection chamber 206 is formed around the lower lamp 216b to separate the upper lamp 216a and the lower lamp 216b. A power distribution block is housed on top of the fixture 200 within a 4″×4″ square junction box 212, as shown in FIG. 15, which is also provided for power and control wiring connections. The purpose of the power distribution block is to comply as a UL-listed luminaire disconnect to connect to input power wiring for maintenance, in compliance with the National Electrical Code (NEC). The junction box 212 includes four (4) ½″ knockouts 214, two (2) per opposite sides, for conduit and wire entry and is positioned in the center rear of the fixture 200.

As shown in FIGS. 10-12, four aluminum grills 210 are provided on each side of the housing 202. The grills 210 allow for UV-C light to emit from the fixture 200 for the purpose of Upper Room operation. The aluminum grills 210 are orientated at ninety degrees for the purpose of shielding any downward emitted light from direct view. For this purpose, the fixture 200 is to be mounted at heights of no less than 8 feet in order to avoid direct view of the Upper Room UV-C lamp's 216a operation during occupancy. The aluminum grills 210 are constructed of aluminum and painted black but one skilled in the art will recognize that other materials and colors could be used.

Referring to FIGS. 11 and 12, eight frictionless fans 224 are operably mounted on every corner of the housing 202. In this embodiment, the fans 224 are mounted adjacently, two per corner of the fixture 200. All eight fans 224 rotate in the same direction to expel air from the fixture 200 for the purpose of improving air mixing within the room which will improve Upper Room UV-C treatment efficacy. One of ordinary skill in the art would recognize that the number of fans 224 could be modified without departing from the spirit and scope of the invention.

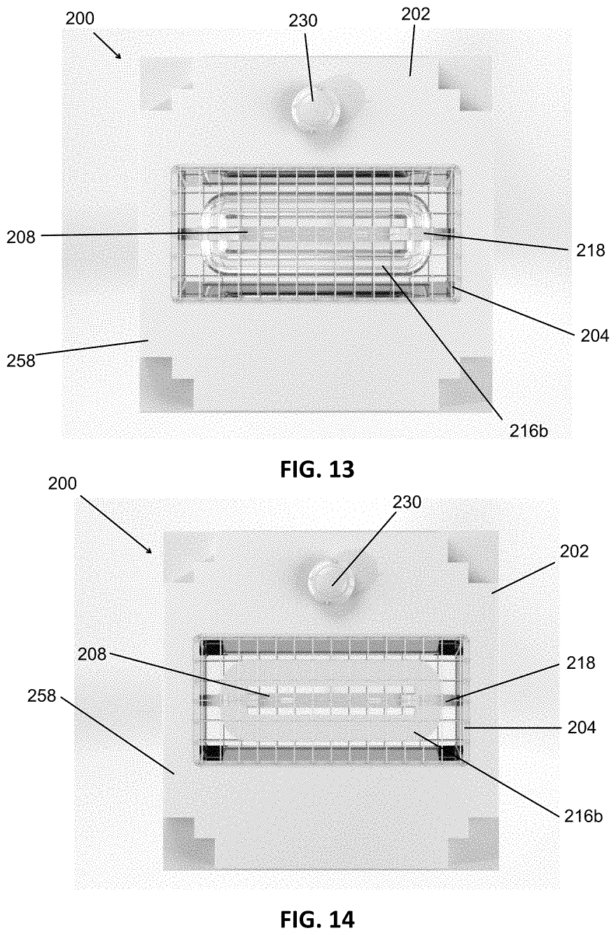

Referring to FIG. 11, various components of the fixture 200 are secured to the interior of the housing 202. Two 21.7″×6.7″ UV-C lamps 216a, 216b are mounted to four UV-C resistant ceramic lamp holders 218. As shown in FIGS. 13 and 14, the ceramic lamp holders 218 are mounted directly to a stainless-steel support bar 208, which extends between opposing ends of the lamp 216a, 216b. The lamps 216a, 216b operate at high temperature and as such, the ceramic lamp holders 218 offer thermal protection. Each ceramic lamp holder 218 features an integrated 4-pin electrical connector and wire harness for the lamps 216a, 216b to connect to. In this embodiment, the two UV-C lamps 216a, 216b are induction mercury UV-C lamps with specifications as follows—(i) size: 21.7″ (L)×6.7″ (W)×4″ (D); power: 300 W; (iii) voltage: AC120-277V; (iv) UV strength: ≥1,300 micro watt per square centimeter at 3.5 feet; (v) effective volume: 70,500 cubic feet; (vi) UVC wavelength: 253.7 nm; (vii) lamp material: Amalgam Quartz; (viii) IP Rating: IP65; and (ix) bulb lifespan: ≥60,000 hours. Each of the UV-C lamps 216a, 216b are operably coupled to two electronic ballast 220 by wire. In this embodiment, the two ballast 220 are suitable for a 300 W 1-lamp load, operational for 100-277 VAC and rated for duty cycling. The UV-C lamps 216a, 216b and UV-C ballasts 220 are commercially available components and are not proprietary. The purpose is to allow users of fixture 200 to be able to easily source replacements parts after said components reach end of lifespan. One of ordinary skill in the art would recognize that the number and size of the lamps 216a, 216b and ballast 220 could be modified without departing from the spirit and scope of the invention.

Referring to FIG. 15, four (4) ⅜″ O-Bolts 222 are mounted in each corner on a top surface of the fixture 200 for chain mounting to a ceiling. Additionally, the O-Bolts 222 can be removed and replaced with ⅜″ threaded rods by the installer for an additional mounting method. Alternatively, the fixture 200 could be mounted with a ceiling mounting bracket 226. The ceiling mounting bracket 226 includes bolts 232 for coupling with a top surface of the fixture 200. The ceiling mounting bracket also includes two (2) oval mounting slots 228 to accept bolt sizes up to ½″. The ceiling mounting bracket 226 is for the purpose of mounting directly to the ceiling and/or adjusting the angle of tilt. The two pre-drilled openings or mounting slots 228 are ⅜″ wide and 16″ apart to accommodate standard stud spacing. One of ordinary skill in the art would recognize that these openings could be spaced apart at other lengths, for example, 24 inches, or further openings could be provided to accommodate for alternative mounting.

As shown in FIG. 11, a motion sensor 230 is also mounted to the housing 202 and operably coupled with the controller 250. Preferably, the motion sensor 230 and controller 250 are those manufactured and sold under the mark Intelli-Safe. The motion sensor 230 is a radio frequency (RF) sensor that can detect occupants within the treatment area and maintain Whole Room treatment and/or disengage operation as per programming. In this embodiment, Whole Room treatments will only begin when the motion sensor 230 does not detect any motion for a pre-programmed time i.e. 5 minutes, but other time limits could be set as desired via the user app 300. One skilled in the art would recognize that other types of motion sensors could be used as will be described below.

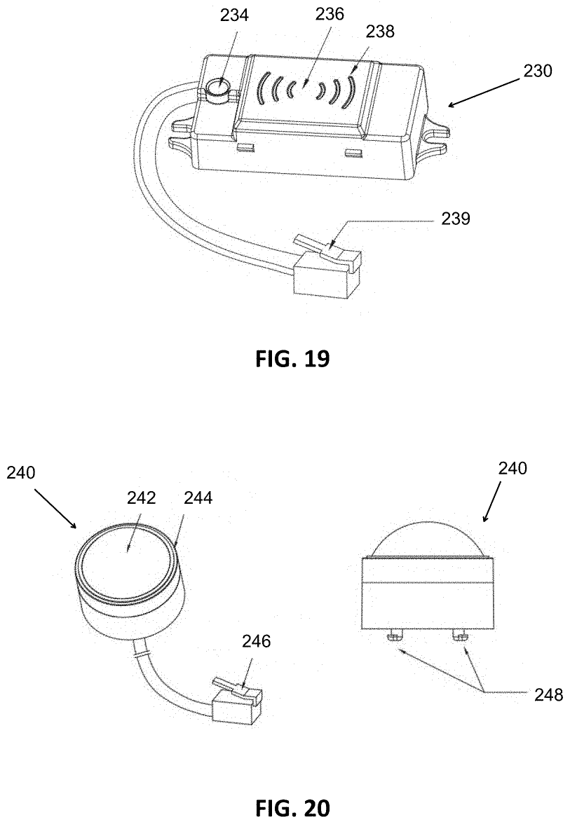

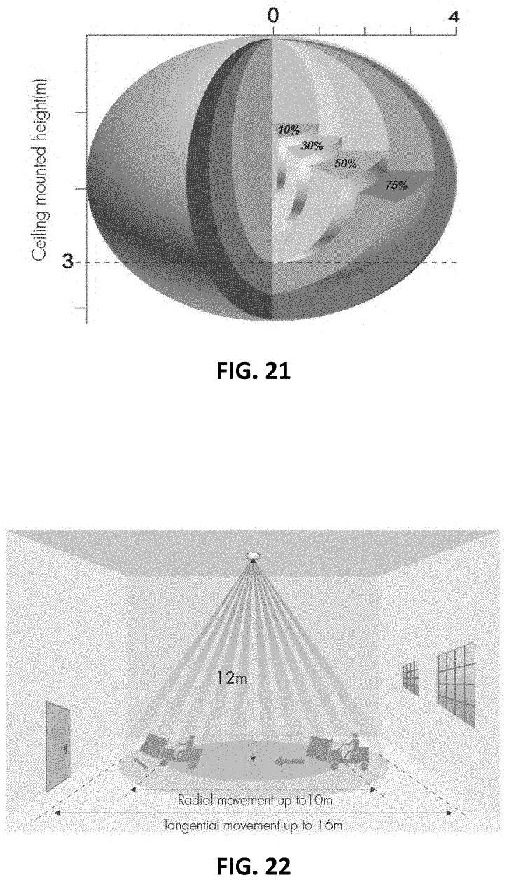

Referring to FIG. 19, the RF sensor 230 includes a daylight sensor 234, a sensor antenna 236 and a Bluetooth module 238. The RF sensor 230 is also provided with an RJ12 connector 239 for coupling with the controller 250, as shown in FIG. 18. The RF sensor 230 includes the following features—(i) sensor principle: high frequency (microwave); (ii) operation frequency: 5.8 GHz+/−75 MHz; (iii) transmission power: less than 0.2 mW; (iv) detection range: Max. (Ø×H) 8 m×3 m (as illustrated in FIG. 21); and (v) detection angle: 30 degrees-150 degrees. However, the detection range is heavily influenced by sensor placement (angle) and different walking paces. The Bluetooth module includes the following features—(i) operation frequency: 2.4 GHz-2.483 GHz; (ii) transmission power: 7 dBm; (iii) range: 10-30 m; and (iv) protocol: Bluetooth 5.0 SIG Mesh.

Referring to FIG. 20, a passive infrared (PIR) sensor 240 could also be used. The PIR sensor 240 includes a lens 242 housed within a housing 244. An RJ12 connector 246 extends from the housing 244 for coupling with the controller 250 and although not shown, the sensor 240 also includes a Bluetooth module having substantially similar features as described above with respect to the RF sensor 230. The sensor 240 includes lugs 248 on a bottom portion for mounting to the fixture housing 202. The PIR sensor 240 includes the following features—(i) sensor principle: PIR detection; (ii) operation voltage: 5 VDC; (iii) detection range: HIR 13×(Ø×H) 16 m×12 m; HIR 16 (L×W×H) 18 m×6 m×15 m (as illustrated in FIG. 22 based on 5 km/h movement speed); and (iv) detection angle: 360 degrees. However, the detection range is heavily influenced by sensor placement (angle) and different walking paces.

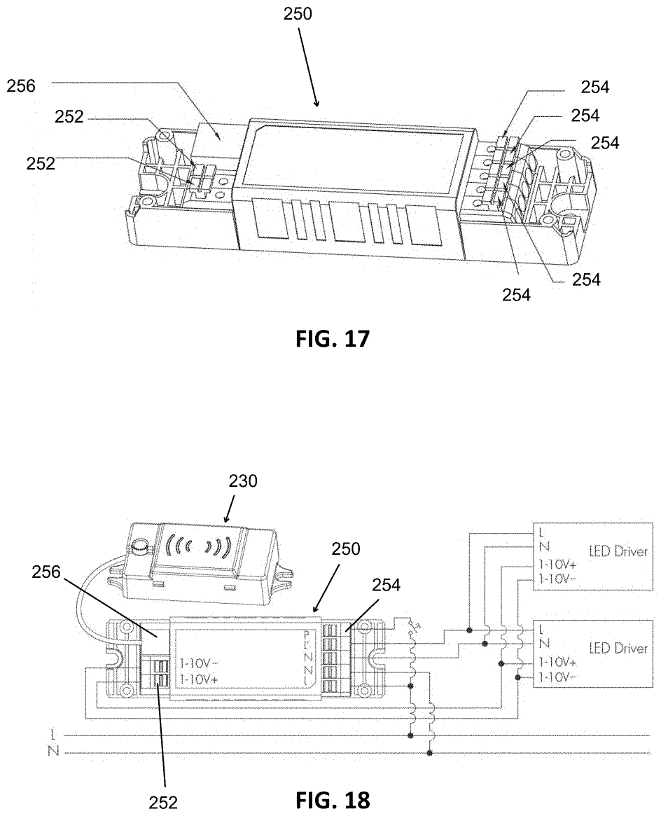

Referring to FIGS. 17 and 18, the controller 250 includes a first set of wire connectors 252 for coupling with a power supply, as well as a second set of wire connectors 254 for coupling with the various components within the fixture 200, as shown in an exemplary wiring diagram in FIG. 18. The controller 250 is also provided with a RJ12 connector 256 for coupling with the sensor 230, 240.

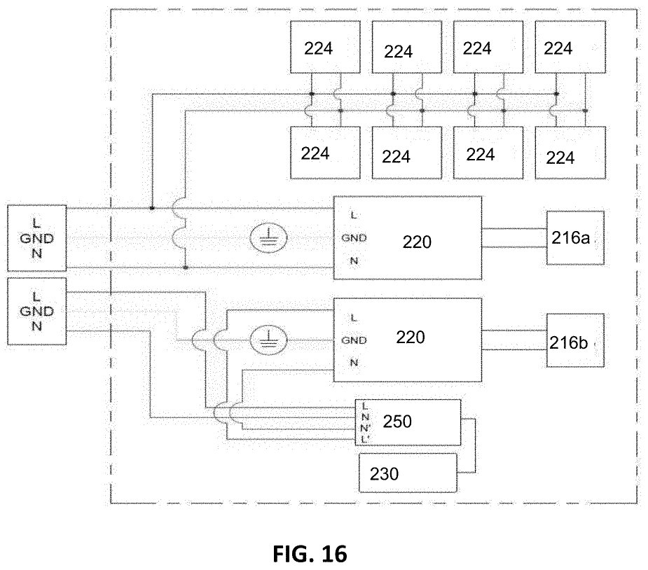

Referring to FIG. 16, the controller 250 is operably coupled by wiring with the sensor 230, one electronic ballast 220 and one UV-C lamp 216b for Whole Room operation. On the other hand, one electronic ballast 220 and one UV-C lamp 216a for Upper Room operation is connected to eight fans 224 and are operated via an external line-voltage switch, such as a light switch located in the room in which the fixture 200 is located. Input wiring connections are made within the junction box 212 at the power distribution block. The controller 250 is programmed and controlled by the user via the user app 300. In this embodiment, the user app 300 operates on iOS and Android. However, the user app 300 could also be used on desktop and laptop computers.

The controller 250 and the user app 300, collectively, is a Bluetooth mesh control system that controls the fixture 200 by executing pre-programmed and on-demand automated disinfection in order to ensure the Whole Room treatment mode is in accordance with the various features, including the exemplary features outlined above and explained in more detail below. The control system, i.e., the controller 250 and user app 300, which applicant markets under the trademark Intelli-Safe, includes many features that aid in safely disinfecting an interior environment. A remote monitoring wireless hub may be provided with the system 1 of the present invention so that the control system is connected to the user's computer network 20. As such, the user will have the ability to remotely monitor status and operation of the fixture 200.

### Operation of System with Fixture of Second Embodiment

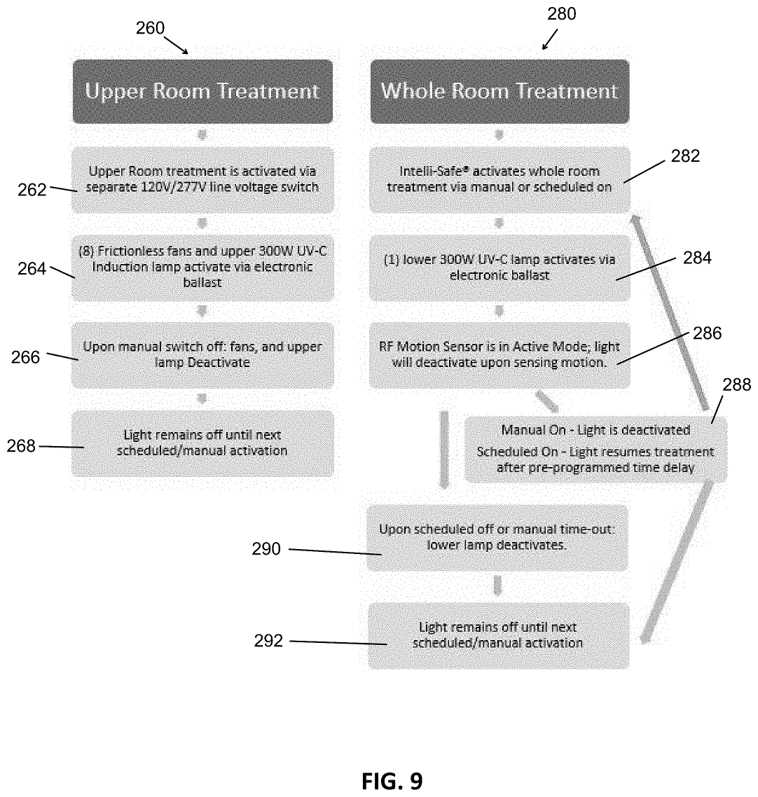

Referring to FIG. 9, in operation, once the Upper Room treatment mode 260 has been initiated via a manual switch (Step 262), eight frictionless fans 224 and one upper 300 W UV-C induction lamp 216a will activate (Step 264). Preferably, the fixture 200 operates at a wavelength of 254 nm but the wavelength could be varied based on utilized UV lamp technology. In order to prevent any UV-C irradiation from direct eye visibility, fixtures 200 are mounted at no less than 8 ft heights from the floor in order to ensure that output threshold limits as a result of direct eye exposure are not exceeded. Upon the termination of the Upper Room treatment, one Upper Room lamp 216a and eight fans 224 deactivate (Step 266) and remain off until the next manual activation (Step 268).

Still referring to FIG. 9, when Whole Room treatment 280 is initiated via the controller 250, a timed treatment will occur in accordance with the pre-programmed settings in the user app 300 (Step 282). The lower UV-C lamp 216b will be on (Step 284). The Hybrid UV-C fixture 200 will turn off once the Whole Room timed treatment ends in accordance with the controller 250 programming. As well, if any motion is detected by the motion sensor 230 during the Whole Room treatment (Step 286), then the fixture 200 will immediately deactivate for a preset period of time during which no motion is sensed (Step 288). During this situation, once the motion time-out period expires the fixture 200 will resume Whole Room mode (Step 282) to finish the current treatment or deactivate in accordance with the controller 250 programming (Step 290). Once the scheduled or manual operation time treatment completes then each of above-mentioned components remain off until the next scheduled activation or manual activation (Step 292).

### Portable Fixture Embodiments

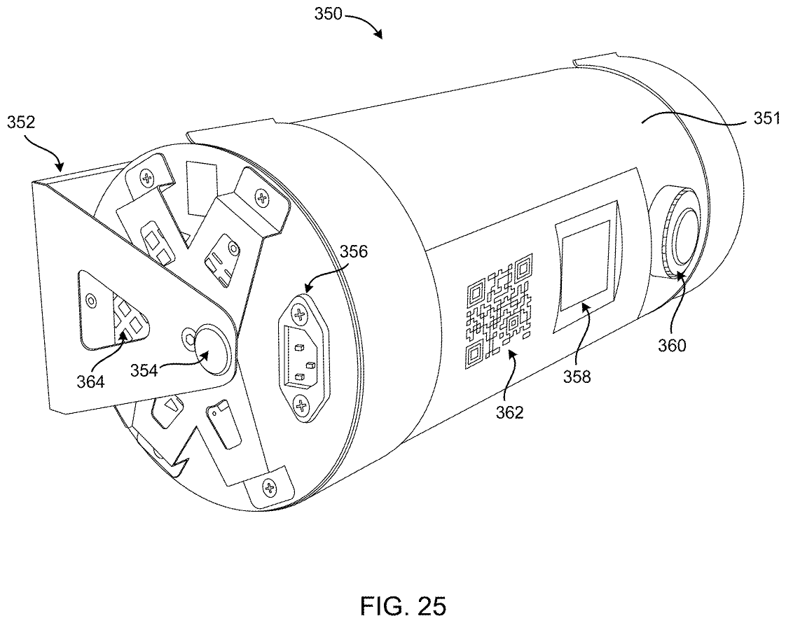

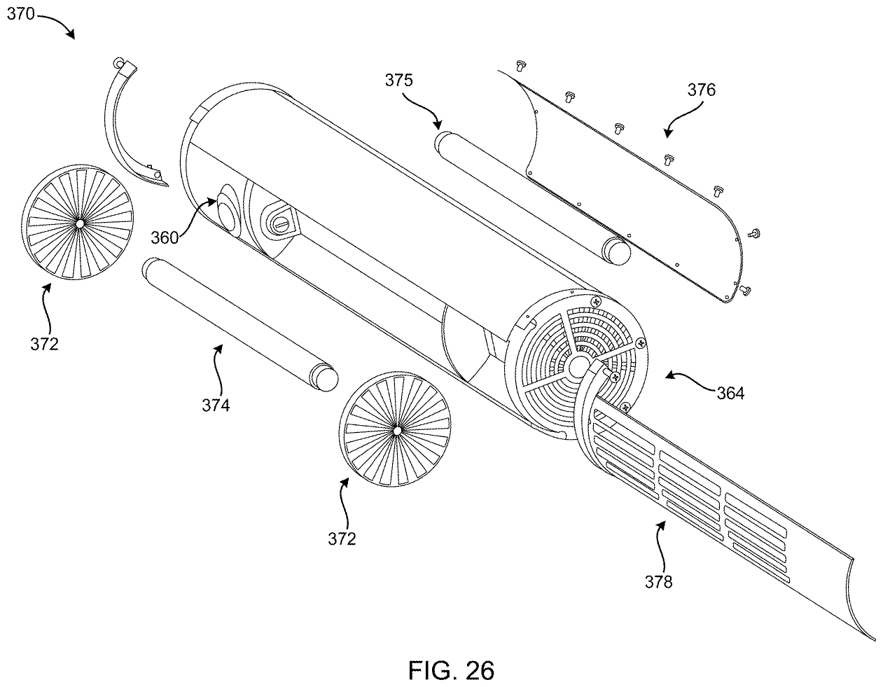

Referring to FIGS. 25 and 26, additional embodiments of a fixture 350, 370 of the present invention is shown. The fixtures 350, 370 are portable units that can be used in smaller spaces such as inside an automobile. The fixtures 350, 370 are especially useful for enclosed spaces that require clean air and clean surfaces such as the inside of an ambulance. As with the other fixture embodiments described herein, the portable fixtures 350, 370 are also hybrid in that both Upper Room treatment and Whole Room treatment are available. The portable fixtures 350, 370 feature an enclosed upper air treatment compartment with fan circulation for the purpose of eliminating any unwarranted exposure of UVC radiation during occupancy treatments. The portable fixtures 350, 370 also provide whole room treatment in different form, as will be described below.

Referring to FIGS. 25, the portable fixture 350 includes an enclosure 351 enclosing internal components of the fixture 350. The enclosure 351 and wiring can vary from model to model in order to fit the need for it to be easily transportable and to maintain portable operation. A mounting bracket 352 is provided for mounting the fixture 350. The mounting bracket 352 is rotatably coupled to the fixture 350 with a swivel member 354 such as a screw to enable the fixture 350 to be positioned as desired by the user depending on how and where the fixture 350 is mounted, e.g., horizontal or vertical surface. Alternatively, the mounting bracket 352 could be fixed to the fixture 350. The fixture 350 is provided with a plug-in power input 356 to accommodate for 110V-277V/60 Hz AC, however, the fixture 350 could be configured to operate under other voltages and frequencies. The fixture 350 also includes a UVC module 358 having at least one UVC lamp for disinfecting and sterilizing the indoor environment during Whole Room treatment during non-occupancy. The UVC lamp of the UVC module 358 could have wavelengths between 100 and 280 nm, but preferably 222 nm or 254 nm. The UVC lamp of the module 358 can consist of mercury, LED, excimer and/or other types of commercially available UVC lamps. An occupancy sensor 360 extends from the enclosure 351 for detecting whether occupants are nearby. The sensor 360 could be PIR, microwave and/or supersonic, or a combination of the same. A QR code 362 is provided for users to access a portal to the control system as will be explained in more detail below. Air vents 364 are provided on opposing ends of the fixture 350 for inlet and outlet of air. Fans are provided on each opposing end of the fixture 350 for providing an air stream from the inlet to the outlet through the air vents 364. A HEPA filter and activated carbon filter is provided downstream from the inlet fan and another HEPA filter and activated carbon filter is provided upstream from the outlet fan for filtering particles in the air flowing therebetween. As well, at least one inner UVC lamp is positioned between the inlet and outlet, preferably far UVC (254 nm) for treating the air circulating through the fixture 350. However, the UVC lamps 374 could have wavelengths between 100 and 280 nm. Moreover, the UVC lamps 374 can consist of mercury, LED, excimer and/or other types of commercially available UVC lamps. With the filter and inner UVC lamp combination, both Upper Room treatment and air purification could be performed simultaneously. As well, when a 222 nm UVC module is used, Whole Room treatment can be activated even during occupancy while performing air purification via the filters. As with the other fixture embodiments described herein, the fixture 350 includes a controller operably coupled with the sensor 360 and the fans are programmed and controlled by the user via the user app 300, as will be described in more detail below. Also, a remote IAQ monitor 504, as described in more detail below, could also be operably coupled with the controller for controlling air purification.

Referring to FIG. 26, in this embodiment, the portable fixture 370 is shown without a mounting bracket for purposes of clarity. In this embodiment, the portable fixture 350 is substantially similar to the portable fixture 350 with a few exceptions, which will be explained in more detail below. As with the portable fixture 350 described above, the portable fixture 370 includes a plug-in power input to accommodate for 110V-277V/60 Hz AC, however, the fixture 370 could be configured to operate under other voltages and frequencies. An occupancy sensor 360 is mounted to the fixture 370 for detecting whether occupants are nearby. The sensor 360 could be PIR, microwave and/or supersonic, or a combination of the same. A QR code is provided on an outer surface of the fixture 370 for users to access a portal to the control system as will be explained in more detail below. Air vents 364 are provided on opposing ends of the fixture 370 for inlet and outlet of air. Fans are provided on each opposing end of the fixture 350 for providing an air stream from the inlet to the outlet through the air vents 364. A HEPA filter and activated carbon filter 372 is provided downstream from the inlet fan and another HEPA filter and activated carbon filter is provided upstream from the outlet fan for filtering particles in the air flowing therebetween. As well, two inner UVC lamps 374 are positioned between the inlet and outlet, preferably far UVC (254 nm) for treating the air circulating through the fixture 370. However, the UVC lamps 374 could have wavelengths between 100 and 280 nm. Moreover, the UVC lamps 374 can consist of mercury, LED, excimer and/or other types of commercially available UVC lamps. In this embodiment, one of the inner UVC lamps 374 is covered with a front plate 376 for use during occupancy, i.e., Upper Room treatment. The other inner UVC Lamp 374 is covered with a back plate 378 having vents for use during non-occupancy, i.e., Whole Room treatment. With the filter 372 and inner UVC lamp 374 combination, both Upper Room treatment and air purification could be performed simultaneously. Moreover, as with the embodiment in FIG. 25 described above, this embodiment could also include the UVC module 358 for both Whole Room treatment when unoccupied (254 nm) and when occupied (222 nm). As with the other fixture embodiments described herein, the fixture 370 includes a controller operably coupled with the sensor 360 and the fans and is programmed and controlled by the user via the user app 300, as will be described in more detail below. Also, a remote IAQ monitor 504, as described in more detail below, could also be operably coupled with the controller for controlling air purification.

### High Ceiling Fixture Embodiment

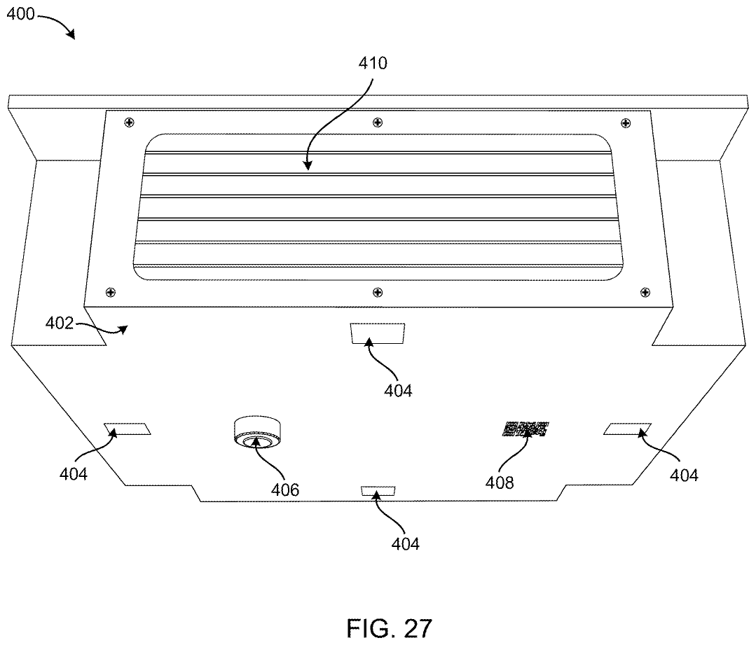

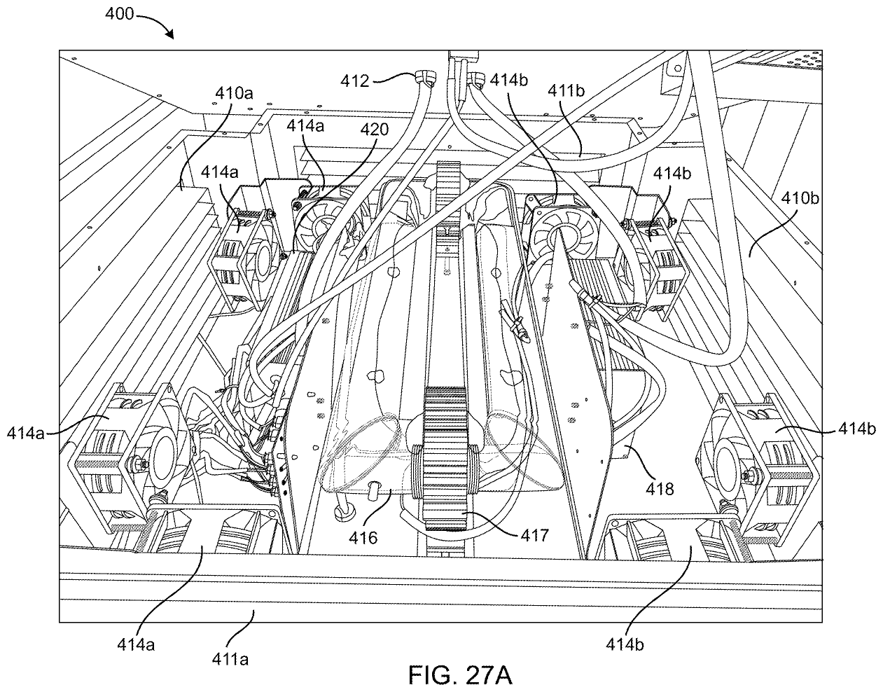

Referring to FIGS. 27 and 27A, an embodiment of a fixture 400 for high ceiling mount is shown. As with the other fixture embodiments described herein, the fixture 400 is also hybrid in that both Upper Room treatment and Whole Room treatment are available. The fixture 400 features an enclosed upper air treatment compartment with fan circulation for the purpose of eliminating any unwarranted exposure of UVC radiation during occupancy treatments. The fixture 400 also provides whole room treatment via UVC modules as will be described below. The fixture 400 includes an enclosure 402 enclosing internal components of the fixture 400. A top portion of the enclosure 402 is mounted to a ceiling of an interior environment such as a room or interior of a vehicle. The fixture 400 can provided with a plug-in power input 412 to accommodate for 110V-277V/60 Hz AC, however, the fixture 400 could be configured to operate under other voltages and frequencies. Alternatively, the fixture 400 could be hard-wired to a power source. A bottom portion of the fixture 400 includes a plurality of UVC modules 404 each having at least one UVC lamp for disinfecting and sterilizing the indoor environment during Whole Room treatment during non-occupancy as well as Whole Room treatment during occupancy. The UVC lamps of each downwardly facing UVC module 404 could have wavelengths between 100 and 280 nm, but preferably 222 nm or 254 nm. The UVC lamp of the module 404 can consist of mercury, LED, excimer and/or other types of commercially available UVC lamps. As described above, when a 222 nm UVC module is used, Whole Room treatment can be activated even during occupancy. When a 254 nm UVC module is used, Whole Room treatment can be activated only when unoccupied. An occupancy sensor 406 extends from the bottom portion of the enclosure 402 for detecting whether occupants are nearby. The sensor 406 could be PIR, microwave and/or supersonic, or a combination of the same. A QR code 408 is provided for users to access a portal to the control system as will be explained in more detail below. A pair of opposing air vents 411a, 411b are provided on a front and rear portion of the enclosure 402, respectively, and a pair of opposing air vents 410a, 410b are provided on a left and right portion of the enclosure 402 are for inlet and outlet of air. In this embodiment inlet fans 414a are positioned and mounted behind the front, left and rear vents 411a, 410a, 411b and outlet fans 414b are positioned behind the front, right and rear vents 411a, 410b, 411b. In this embodiment, the inlet fans 414a are configured to draw air into the enclosure 402 and the outlet fans 414b are configured to draw air out of the enclosure 402. A HEPA filter and activated carbon filter is provided between within each fan 414a,414b for filtering particles in the air flowing therebetween. Alternatively, the filters could be positioned on the vents 410a, 410b, 411a, 411b. As shown in FIG. 27A, the enclosure 402 is provided with four vents and eight fans to provide sufficient air flow within the enclosure 402. However, the enclosure 402 could also be configured to have less vents and less fans. As well, at least one inner UVC lamp 416 powered by an inner lamp ballast and driver 418 is positioned within the enclosure 402, and secured with an inner lamp holder 417. In this embodiment, the inner UVC lamp 416 is preferably far UVC (254 nm) for treating the air circulating through the fixture 400. However, the inner UVC lamp 416 could have wavelengths between 100 and 280 nm. Moreover, the inner UVC lamp 416 can consist of mercury, LED, excimer and/or other types of commercially available UVC lamps. A separate ballast and drive 420 for the UVC modules 404 is also positioned within the enclosure 402. As with the other fixture embodiments described herein, the fixture 400 includes a controller operably coupled with the sensor 406 and the fans 414a, 414b and is programmed and controlled by the user via the user app 300, as will be described in more detail below. Also, a remote IAQ monitor 504, as described in more detail below, is operably coupled with the controller for controlling air purification. With the filter and inner UVC lamp 416 combination, both Upper Room treatment and air purification could be performed simultaneously. Moreover, Whole Room treatment could be performed simultaneously with air purification when unoccupied (using 254 nm UVC module) and when occupied (using 222 nm UVC module).

### Low Ceiling Fixture Embodiment

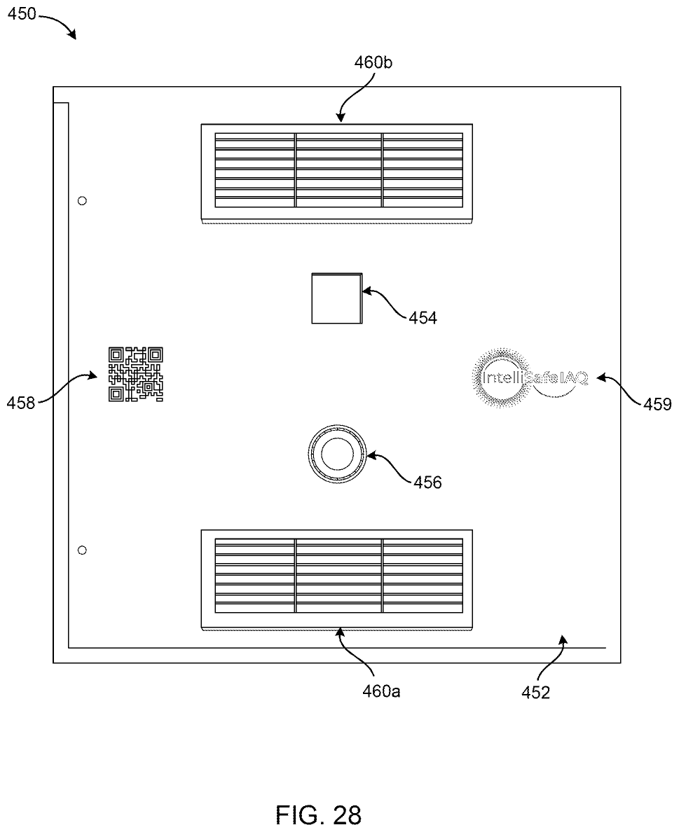

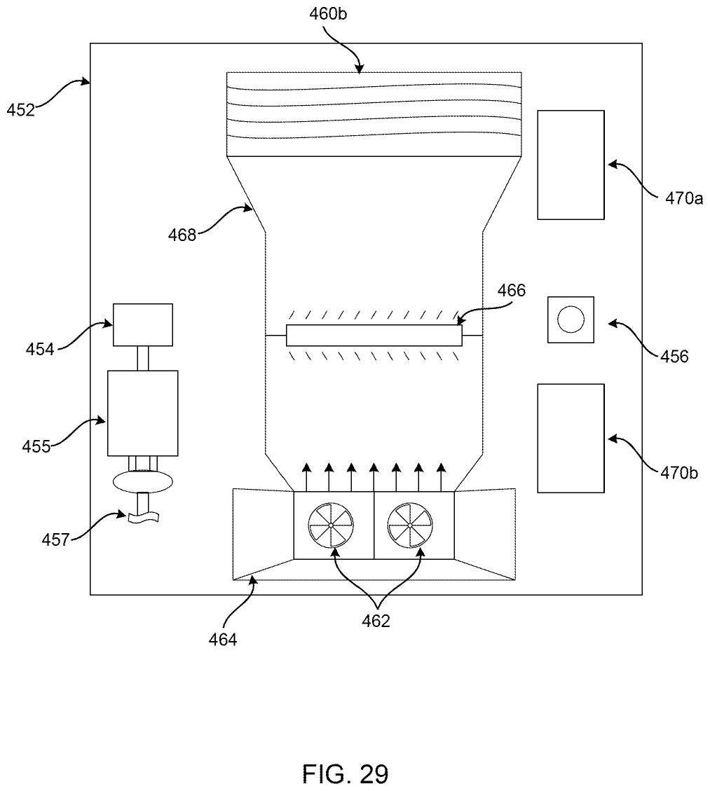

Referring to FIGS. 28 and 29, an embodiment of a fixture 450 for low ceiling mount is shown. As with the other fixture embodiments described herein, the fixture 450 is also hybrid in that both Upper Room treatment and Whole Room treatment are available. The fixture 450 features an enclosed upper air treatment compartment with fan circulation for the purpose of eliminating any unwarranted exposure of UVC radiation during occupancy treatments. The fixture 450 also provides whole room treatment via UVC modules as will be described below. The fixture 450 includes an enclosure 452 enclosing internal components of the fixture 450. A top portion of the enclosure 452 is mounted to a ceiling of an interior environment such as a room or interior of a vehicle. The fixture 450 can provided with a plug-in power input to accommodate for 110V-277V/60 Hz AC, however, the fixture 450 could be configured to operate under other voltages and frequencies. Alternatively, the fixture 450 could be hard-wired to a power source. A bottom portion of the fixture 450, as shown in FIG. 28, includes at least one UVC module 454 having at least one UVC lamp for disinfecting and sterilizing the indoor environment during Whole Room treatment. The UVC lamps of each downwardly facing UVC module 454 could have wavelengths between 100 and 280 nm, but preferably 222 nm or 254 nm. The UVC lamp of the module 454 can consist of mercury, LED, excimer and/or other types of commercially available UVC lamps. As such, Whole Room treatment is capable of being performed during non-occupancy (when 254 nm is used) as well as during occupancy (when 222 nm is used). The UVC module 454 is operably coupled with a driver 455 which is operably coupled to a connecting harness 457 for coupling to a controller. An occupancy sensor 456 extends from the bottom portion of the enclosure 452 for detecting whether occupants are nearby. The sensor 456 could be PIR, microwave and/or supersonic, or a combination of the same. A QR code 458 is provided for users to access a portal to the control system as will be explained in more detail below. Optionally, a logo 459 could be affixed to the enclosure 452 for marketing purposes. Air vents 460a, 460b are provided on the bottom portion of the enclosure 452 are for drawing in air and discharging air. At least one fan 462 is provided behind the inlet air vent 460a and mounted within the enclosure 452. However, the fixture 450 could also be configured to have less or more fans. In this embodiment, each fan 462 is variable speed and configured to operate at 0-500 CFM. A HEPA filter is provided on the inlet air vent 460a and an activated carbon filter 464 is provided on the outlet air vent 460b for filtering particles in the airstream entering and exiting the enclosure 452. As well, an inner UVC lamp 466 is positioned within an air treatment chamber 468 downstream from the fans 462, preferably far UVC (254 nm) for treating the air circulating through the fixture 450. However, the UVC lamp 466 could have wavelengths between 100 and 280 nm. Moreover, the UVC lamp 466 can consist of mercury, LED, excimer and/or other types of commercially available UVC lamps. As well, more than one UVC lamp could be provided depending on the volume of airflow. As with the other fixture embodiments described herein, the fixture 450 includes a controller operably coupled with the sensor 456 and the fans 462 and is programmed and controlled by the user via the user app 300, as will be described in more detail below. The electrical components, i.e., ballasts, drivers, wireless smart controls and fan speed control, are housed within at least two inner compartments 470a, 470b. Also, a remote IAQ monitor 504, as described in more detail below, is operably coupled with the controller for controlling air purification. With the filter and inner UVC lamp 416 combination, both Upper Room treatment and air purification could be performed simultaneously. Moreover, Whole Room treatment could be performed simultaneously with air purification when unoccupied (using 254 nm UVC module) and when occupied (using 222 nm UVC module).

### Control System and User App

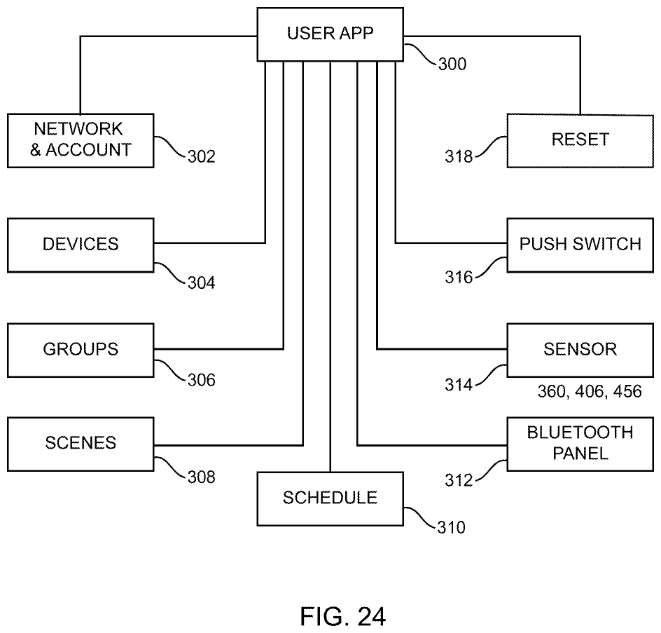

Referring to FIG. 24, numerous features and parameters are programmed in the controllers, e.g., 150, 250, via the user app 300 specific to the type of fixture 100, 200, 350, 370, 400, 450. The user app 300 is provided with GUIs corresponding to the numerous features and parameters for the user to select and edit. In one aspect, there are three levels of users for the user app 300: owner, installer and sub-user, which are programmed by selecting a “Network & Account” feature 302 of the user app 300. The owner is the primary user for the network and one network belongs to only one owner. The owner can control all installers and sub-accounts associated with a network. If the owner deletes the network, the network will then be removed from all the installers and sub-accounts. The owner can share the network with the installer to do commissioning work. One network can have many installers. When an installer deletes the network which is associated with multiple accounts, this same network will not be removed from other accounts, meaning that the installer cannot control other accounts associated with this network. Otherwise, the installer and owner have the same access permissions to the network. The sub-user, meaning the guest user, can use the network normally, but for some operations such as deleting the network, sharing the network to others, etc., the sub-user requires permission from the owner. Networks can be shared with a QR code or keycode, and different permission levels can be set by the network owner. The user is also able to add specific fixtures 100, 200, 350, 370, 400, 450 to manage by selecting a “Devices” feature 304 on the user app 300.

In another aspect, the user app 300 also provides different fixtures 100, 200, 350, 370, 400, 450 to be grouped through a “Groups” feature 306. Grouping ensures that multiple enabled Hybrid UV-C fixtures 100, 200, 350, 370, 400, 450 operate in accordance with the same pre-programming. Correspondingly, the grouping feature can deactivate all hybrid UV-C fixtures 100, 200, 350, 370, 400, 450 within a specific zone when only one RF sensor is activated. One luminaire or fixture 100, 200, 350, 370, 400, 450 can belong to many different groups, regardless of the other luminaries in the same group being from different rooms. For example, luminaire A in room 1 can group with luminaire B in room 2. All fixtures 100, 200, 350, 370, 400, 450 in the same group will be linked together automatically, allowing all fixtures 100, 200, 350, 370, 400, 450 within the same group to work together. As such, once a single motion sensor, e.g., 130, 230, has been triggered, the others will be triggered at the same time, thus, synchronizing control.

In yet another aspect, scenes can be programmed via the user app 300 by selecting a “Scenes” feature 308. Scenes are a very useful and important function to the user. Users can create a variety of customized scenes through this feature. The scene can be recalled by the motion sensor, scheduling, push switch and/or Bluetooth panel features within the user app 300. Many types of scenes are available for users to program. In one embodiment, initially, three default scenes are available: all on, 50% on, and all off. Users can create up to 16 scenes for a single fixture 100, 200, 350, 370, 400, 450, for example, generic scene, lux on/off scene, daylight harvest scene, circadian rhythm scene and time-based scene.

The user app 300 also provides a scheduling feature, which the user could set and modify by selecting a “Schedule” feature 310. The scheduling function is another important feature for the users. With this function, the user can create a list of timers that will turn scenes on and off based on time. For example, the user can set a luminaire or fixture 100, 200, 350, 370, 400, 450 to activate during office hours, non-office hours or set corridor lights dim to a lower level at night. The user can also set a schedule based on an Astro timer (sunrise and sunset). The astronomic scheduling feature ensures that the Hybrid UV-C fixture 100, 200, 350, 370, 400, 450 operates during a pre-defined schedule and deactivates when treatments are not required. For example, a thorough long-term Whole Room treatment can be activated every night between 1 AM-2 AM for a comprehensive hands-off viral, bacterial and fungal disinfection on a routinely scheduled basis. The calendar function can be synced to the main user's IOS/Android device in order to ensure that time is accurate.

In another aspect, on-demand disinfection can be initiated by authorized users at any time via app-based controls. In one feature, the user app 300 is provided with a Bluetooth panel 312. With this feature, the user is capable of controlling the fixtures 100, 200, 350, 370, 400, 450 wirelessly. The user could also commence manual activation via the user app 300. In one embodiment, manual activation of Whole Room treatment will automatically and immediately revert to Upper-Room treatment if any motion is detected within the treatment area as an added safety measure. Alternatively, Whole Room treatment could be activated manually via the user app 300.

In another feature, the user app 300 is provided with a virtual wall switch or push switch 316. For example, the switch could activate or deactivate a particular fixture 100, 200, 350, 370, 400, 450, room or group for Upper Room or Whole Room treatment.

The user app 300 also provides settings for the UV lamps, e.g., 116, 216a, 216b, which can be set and modified through the “Bluetooth Panel” feature 312. The user can change the brightness and color temperature for the luminaires or fixtures 100, 200, 350, 370, 400, 450. In one embodiment, the user app 300 provides two types of dimming: linear or logarithm. Normally, this dimming profile should be in line with the dimming pattern of Dali drivers. The user can change the load type as well. For example, it can be dimming only, or both dimming and color temperature tuning. Also, the maximum and minimum brightness and color temperature could be adjusted. Furthermore, a “status after repowered” parameter is provided to allow the user to set status of the luminaire after repowering. This is very useful for accidental power shut down. The user can choose it to remain off, stay at customized brightness and color temperature or just recover to the status before powered off. As well, method of manual mode exit could be set to program if, when and how controls will revert back to sensor control.

The user app 300 also provides settings for the sensors, e.g., 130, 230, 240, of the fixtures 100, 200, 350, 370, 400, 450 through a “Sensor” feature 314. For example, the user could select a particular sensor, e.g., 130, 230, 240, to control particular fixtures 100, 200, 350, 370, 400, 450, rooms or groups, based on motion or daylight. As another example, within the sensor controls, the user is capable of further programming activations of scenes under various other parameters such as auto, semi-auto, priority and staircase function, as well as adjusting the sensitivity of the RF sensor from 10%-100% via 10% increments.

Other programmable features of the user app 300 include configuring floor plans to simplify project planning, off-line commissioning, remote control via gateway support HBGW01 and device firmware update over-the-air (OTA).

In one embodiment, the fixture 100, 200, 350, 370, 400, 450 is provided with an emergency back-up. That is, the fixture 100, 200, 350, 370, 400, 450 is programmed to deactivate in case of power loss or controller failure. The controller, e.g., 150, 250, includes a built-in memory function to retain pre-programmed settings via solid state memory up to 12 weeks. The system is capable of being reset as well, through the “Reset” feature 318.

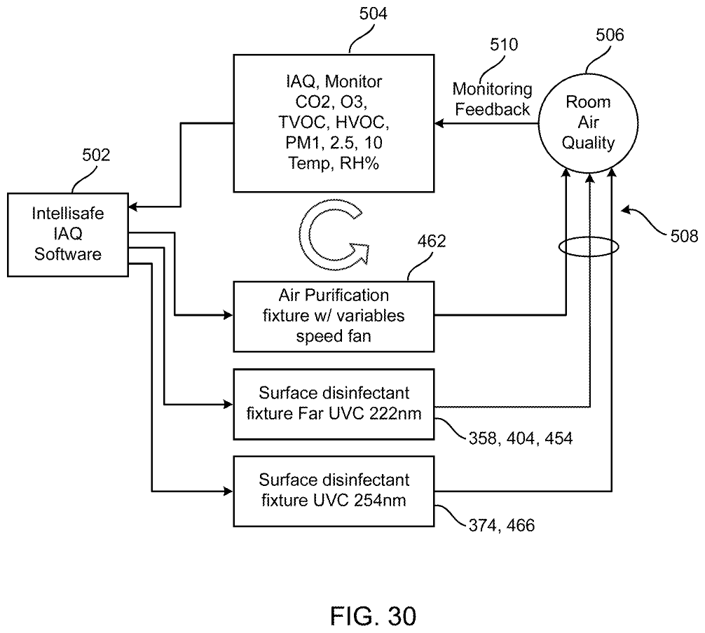

Referring to FIGS. 30-34, another embodiment of the control system of the present invention is illustrated. This embodiment may incorporate the features of the system and user app 300 described above, and provides additional features specifically used for the fixtures 350, 370, 400, 450 having UVC lamps, UVC modules, and HEPA and carbon filters. As discussed above, this embodiment provides a one-of-a-kind holistic approach to ensuring that healthy air quality is maintained via IAQ and UVC technologies and provides validation via real-time data and occupant peace-of-mind and confidence via direct engagement with multiple user-interface options. Healthy air quality is maintained via a real-time feedback loop using indoor air sensor data to ensure that healthy air thresholds are achieved. Unlike other technologies that focus exclusively on limiting particulate matter, this system emphasizes use of IAQ and UVC technologies to limit opportunity for pathogen and particulate spread while remaining flexible for application to devices additionally utilizing other filtration technologies including HEPA, active carbon, and ventilation.

Referring to FIG. 30, in general, software 502 commercially known as Intellisafe IAQ is integrated with controllers of a fixture 350, 370, 400, 450 and more specifically to the fans, e.g., 462, UVC lamps, e.g., 374, 466, and UVC modules, e.g., 358, 404, 454, of the fixture 350, 370, 400, 450. The controllers of the fixture 350, 370, 400, 450 are coupled to an IAQ monitor 504, which monitors air quality 506 in real time. The fixture 350, 370, 400, 450 is activated and de-activated as shown in the arrows labeled 508 based on user settings and feedback 510 to the IAQ monitor as shown in the arrow labeled 510. When the room air quality is determined healthy, the fixture 350, 370, 400, 450 operates in eco-mode. As such, the system operates in a feedback loop to activate and de-activate the fixture 350, 370, 400, 450. The feedback loop provides quiet and eco-friendly operation of air quality improving technologies when air quality is deemed healthy, and signals air quality improving technologies to address unique needs of individual spaces. As a result, this does not cause entire system to increase operation when only one room needs to be treated and air quality improving technologies can operate different aspects to address different needs, e.g., UVC whole-room disinfection when viral index is high. As well, validation of air treatment via live air quality monitoring is provided. Real-time data can be accessed via multiple user interfaces including a web application, IAQ monitor and a mobile application. In addition to air quality data, the following information is accessible: historic data and reports, component replacement notifications; feedback loop data threshold triggers; on-demand and scheduled treatments; and theme customization including facility logos and color schemes. Unlike other systems in the prior art, this solution provides opportunities for all occupants, e.g., administrators, managers, sub-users and guests, to directly engage with the system with varying levels of access. Unique to this solution, guest access allows all occupants to engage directly with the system via requests to increase/decrease hardware operation, notifying the system of noticeable poor air quality, e.g., bad smell or a constantly coughing occupant, and learning about the science and purpose of the air improvement system. This guest-access importantly provides crucial feedback to building managers and administrators, improves the functioning of the system, and provides holistic physical/mental/emotional wellbeing improvements to occupants. While the system provides some degree of guest interaction, permissions set by administrators set the degree of engagement.

The hardware controls of the system generally includes the fixture 350, 370, 400, 450 and the IAQ monitor 504. As described above, each fixture 350, 370, 400, 450 includes a controller that creates localized mesh network, receives and transmits data between devices or fixtures 350, 370, 400, 450 and the IAQ monitor 504, and receives local room instructions based on IAQ monitor sensor array data. As examples, the far-UVC module, e.g., 358, 404, 454, of the fixture 350, 370, 400, 450 is activated when occupancy is detected and fan speed is increased when PM value exceeds set thresholds due to particle pollutant infiltration. As described above, each fixture 350, 370, 400, 450 also includes an occupancy sensor, e.g., 360, 406, 456, and QR code, e.g., 362, 408, 458. The QR code leads to a webapp portal that provides occupant engagement by displaying information such as the “Science Behind System” with an explanation of functionality, the room's Healthy Air Score and other engagement options including, but not limited to, requesting greater disinfection and reporting poor air quality. User login is required for occupants to access additional features such as live room data and limited manual operation as per granted permissions, e.g., increase or decrease fan speed.

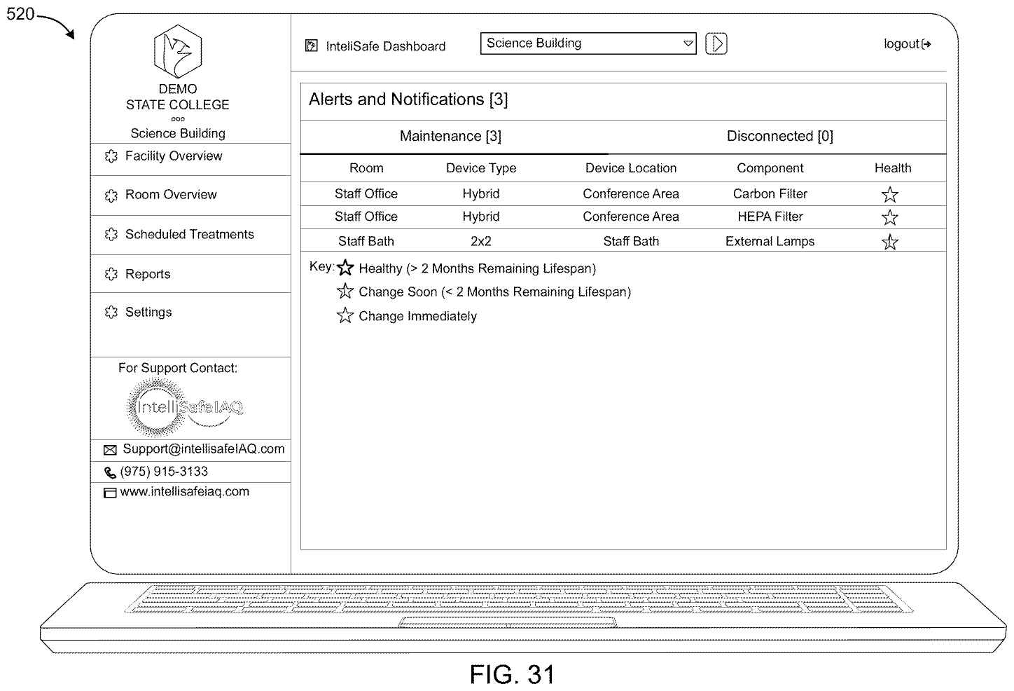

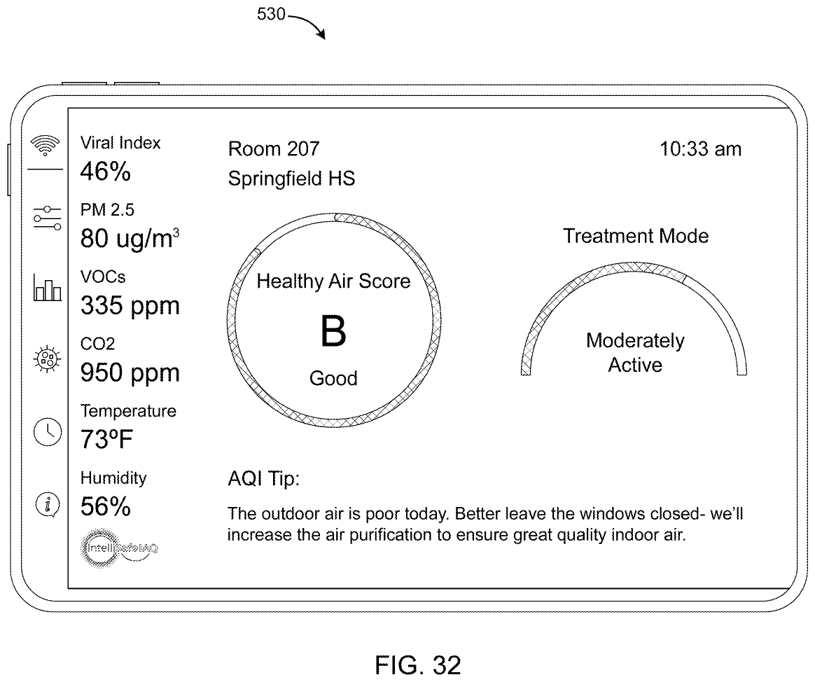

Referring to FIG. 32, with respect to the IAQ monitor 504, the purpose is to provide localized visual air quality data on a digital display 530. A digital screen 530 is displayed with per-area readings for various parameters for a selected room. That is, the IAQ monitor 504 is located remote from the fixtures 350, 370, 400, 450, for example, mounted on a wall within the room the fixture 350, 370, 400, 450 is located, so that the user could set air quality parameters and view real-time air quality using the display 530. The IAQ monitor includes at least one sensor for measuring various air quality parameters. Air score is displayed, which is calculated using a proprietary grade system from weighted values between PM2.5, TVOCs, PM10, CO2, Temperature, and Relative Humidity. Air quality parameters are monitored via sensor and can be accessed and displayed. As well, other system parameters can be accessed and displayed. Those parameters include, but are not limited to, temperature, relative humidity, TVOCs, PM1, PM2.5, PM10, CO2, AQI, UVC dosage, average allergen values, current treatment mode and detection of IoT based devices. The IAQ monitor 504 also displays an AQI Tip, as shown in FIG. 32, to provide users with feedback based on current outdoor air quality. In addition, the IAQ monitor 504 provides germicidal overview, including pathogen remediation information by assigning a grade, e.g., 5-10, for selected pathogens. Reports can also be generated for far UVC treatment detail, pathogen treatment detail. Allergen treatment detail, air treatment detail, air quality detail and device health. Treatment options can be programmed for manual activation, schedules and feedback settings. The user could also configure other settings such as general settings (facility settings, color scheme and saved users), system settings (network, device health check, component replacement, adding and removing devices/fixtures, calibration and far UVC threshold). The IAQ monitor 504 also provides alerts and notifications, as shown for example in FIG. 31, for network connectivity loss, loss of device/fixture connectivity, overdue parts replacement and emergency response, in which case the admin is notified and the system is activated to full treatment mode until the issue is deemed resolved. The IAQ monitor 504 is also provided with contact information for support and a QR code for support tools including YouTube videos and tutorials. The IAQ monitor 504 includes Bluetooth control for local communication protocol via devices and for providing local feedback loop based on live sensor array data. The IAQ monitor 504 is also capable of WiFi connection and provides local AQI via API call, contains a Bluetooth chip to receive and transmit localized data and provides fixtures instructions based on local sensor array data, and serves as a network gateway for Bluetooth network by providing local data to network for remote monitoring and access via an app dashboard 520, as shown in FIG. 31.

Referring to FIG. 31, the purpose of the software 502 is to provide a facility-wide and multi-facility access and control by providing parameters for treatment via the feedback loop, updating of settings, creating and updating users and user system access, providing printable and shareable reports, providing system health check including component replacements notifications, spaces with unusually poor air quality, offline or improperly functioning devices, and firmware version information. The software 502 is integrated with the IAQ monitor 504 and includes display of the information discussed above with respect to the IAQ monitor 504 on the app dashboard 520. The software 502 also provides a facility overview displaying a live treatment map, live average IAQ data including current outdoor air quality index, average facility-wide health score (calculated based on how well the facility meets IAQ thresholds), average viral index (a value estimating the probability of viral transmission based on factors including PM2.5, relative humidity, temperature and CO2), average PM2.5 value, average TVOC value and average allergen value. Also displayed is the 24-hour average germicidal treatment results. These results are based on user selectable virus and bacteria which includes SARS-CoV-2, Influenza, Coxsackievirus, MRSA and C. Diff. A grade score is also determined based on whole-room UVC dosage per device/fixture. The software 502 also provides room overview displaying 24-hour healthy air score and number of devices in the selected room. Other details for each room are provided such data relating to far UVC pathogen, allergen, air treatment, air quality and device/fixture health details. The user is also capable of programming or adjusting local settings for each room such as feedback loop options (sensor triggers, max fan speed and minimum fan speed), treatment schedule (creating new schedules, viewing current schedules and initiating on-demand treatments) and sub-user access (creating and deleted users).

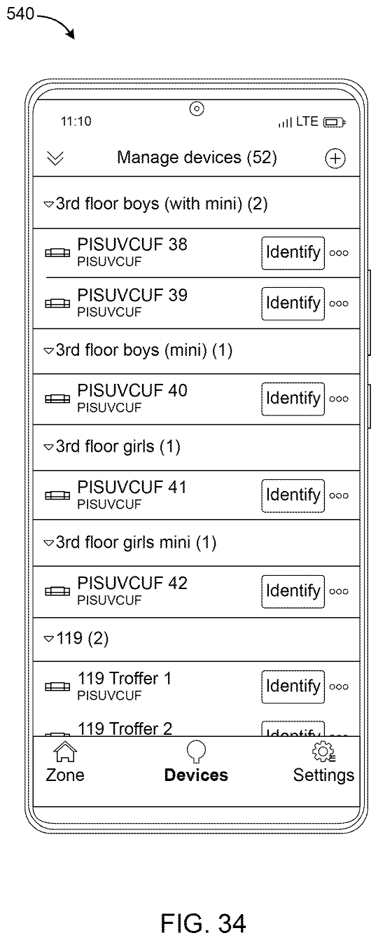

Referring to FIG. 34, a digital display of a commissioning application 540 for the software 502 is shown. The purpose of the commissioning application 540 is to provide a localized Bluetooth application such as iOS or Android to enable a user to commission devices prior to establishing a network. With the commissioning application 540, the user is able to create new projects, edit existing projects and upload shared products created on another user device, among other things. The commissioning application 540 is synced with the cloud to allow usage from multiple user devices and multiple users. In general, the commissioning application 540 is organized by project, network, zone and device/fixture. In the project section of the commissioning application 540, the user selects from existing projects, creates new projects or uploads a shared project, and also selects a network from existing projects. In the network section of the commissioning application 540, zones are created, edited, deleted and tested. Devices/fixtures are added, identified, removed and settings for each device/fixture are entered, e.g., thresholds, sensors and components. As well, the IAQ sensors are calibrated. Additional settings are entered by the user in the network section such as, among other things, access permissions, test mesh for testing signal strength of the network, network settings and local operation settings, e.g., on-demand treatment and schedules.

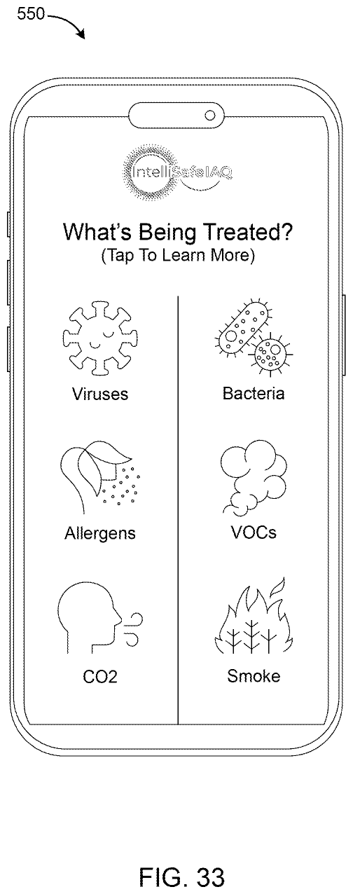

Referring to FIG. 33, a digital display of an occupant engagement web app 550 for the software 502 and system is shown. As discussed above, the purpose of this web app 550 is to provide limited access to local sub-users such as guests to a facility and to engage and educate building occupants. The administrator grants permission to the user and provides a user login. The web app 550 is provides information about the system, company, software and air treatment solutions, for example, viruses, allergens, CO2, bacteria, VOCs and smoke.

The system shown in FIGS. 30-34 and described above provides an interactive feedback loop functionality, live monitoring within a facility's occupied areas, live monitoring of UVC dosage, application of far UVC disinfection with live monitoring loop, capability for customization via client dashboard 520, IAQ monitor 530 and web apps 540, 550, and IoT expandability.

The present invention may be embodied in other specific forms without departing from its spirit or essential characteristics. The described embodiments are to be considered in all respects only as illustrative and not restrictive. The scope of the invention, therefore, will indicated by claims rather than by the foregoing description. All changes, which come within the meaning and range of equivalency of the claims, are to be embraced within their scope.

1. A system for disinfecting an indoor environment, the system comprising:
a user computer having a user app;
a controller operably coupled with the user computer remotely through the user app, the user computer capable of communicating with the controller through a network;
a fixture for housing the controller, the fixture further comprising:
at least one UV-C device operably coupled with the controller,
at least one motion sensor operably coupled with the controller,
at least one fan operably coupled with the controller, and
at least one air filter coupled with the at least one fan; and
at least one air quality sensor;
wherein the at least one UV-C device and the at least one fan are operable in a plurality of modes controlled remotely by a user via the user app.

2. The system of claim 1, wherein the at least one UV-C device includes an inner UV-C device positioned within the fixture and an outer UV-C device positioned on an outer surface of the fixture.

3. The system of claim 2, wherein:
in a first mode, the inner UV-C device and the at least one fan is activated; and
in a second mode, the outer UV-C device is activated; and

4. The system of claim 3, wherein the system is in the first mode when the air quality sensor detects that the quality of air is below a pre-determined threshold or when the motion sensor detects that the indoor environment is occupied.

5. The system of claim 3, wherein the system is simultaneously in the first and second modes when the air quality sensor detects that the quality of air is below a pre-determined threshold and the motion sensor detects that the indoor environment is unoccupied.

6. The system of claim 3, wherein the system is simultaneously in the first and second modes when the air quality sensor detects that the quality of air is below a pre-determined threshold and the motion sensor detects that the indoor environment is occupied.

7. A method for disinfecting an indoor environment, the method comprising the steps of:
programming, by a user computer through a user app located remote from the user computer, parameters for controlling at least one UV-C device and at least one fan having an air filter;
communicating the parameters to a controller, through a network, the controller operably coupled with the at least one UV-C device and a motion sensor for detecting occupancy within the indoor environment, and the at least one fan and an air quality sensor for measuring air quality within the indoor environment;
storing, in a memory, the parameters on the controller; and
controlling, by a processor, the at least one UV-C device and the at least one fan for operation in a first mode when the indoor environment is occupied, a second mode when the indoor environment is unoccupied, and a third mode when air quality parameters are not met;
wherein in the first mode, the at least one UV-C device is activated by the controller to emit ultraviolet radiation to a first section of the indoor environment at a first level, in the second mode the at least one UV-C device is activated by the controller to emit ultraviolet radiation to sections of the indoor environment beyond the first section of the indoor environment at a second level, and in the third mode the at least one fan is activated.

8. The method of claim 7, further comprising the step of:
inactivating, by the controller, the at least one UV-C device in the second mode when the motion sensor detects occupants within the indoor environment.

9. The method of claim 8, further comprising the step of:
re-activating, by the controller, the at least one UV-C device in the first mode when the motion sensor does not detect occupants within the indoor environment for a pre-determined amount of time.

10. The method of claim 9, wherein the at least one UV-C device includes a UV-C lamp and a UV-C module positioned below the UV-C lamp.

11. The method of claim 10, wherein in the first mode only the UV-C lamp is activated by the controller and in the second mode only the UV-C module is activated by the controller.

12. The method of claim 7, wherein the third mode runs simultaneously with the first mode or the second mode.

13. An apparatus for disinfecting an indoor environment, the apparatus comprising:
a controller operably coupled with a remote user computer having a user app, the user computer capable of communicating with the controller via a network;
at least one UV-C device operably coupled with the controller,
at least one motion sensor operably coupled with the controller, and
at least one fan operably coupled with the controller;
wherein the at least one UV-C device and the at least one fan are operable in a plurality of modes controlled remotely by a user via the user app.

14. The apparatus of claim 13, wherein the at least one UV-C device is activated by the controller in a first mode when the motion sensor detects that the indoor environment is occupied, such that the at least one UV-C device emits ultraviolet radiation to an upper section of the indoor environment at a first level for occupants to safely remain within the indoor environment.

15. The apparatus of claim 14, wherein the at least one UV-C device is activated by the controller in a second mode when the motion sensor detects that the indoor environment is unoccupied, such that the at least one UV-C device emits ultraviolet radiation to sections of the indoor environment beyond the upper section at a level greater than the first level.

16. The apparatus of claim 15, wherein the at least one fan is activated by the controller in a third mode when an air quality sensor operably coupled with the controller detects that the quality of air is below a pre-determined threshold.

17. The apparatus of claim 16, further comprising at least one air filter coupled to the at least one fan.

18. The apparatus of claim 14, wherein the activated at least one UV-C device in the first mode is a UV-C lamp.

19. The apparatus of claim 15, wherein the activated at least one UV-C device in the second mode is a UV-C module.

20. The apparatus of claim 16, wherein the third mode runs simultaneously with the first mode or the second mode.
